(19) United States
(12) Patent Application Publication
(10) Pub. No.: US 2025/0000448 A1
(43) Pub. Date: Jan. 2, 2025

(54) AUTOMATIC MEDIA CONTENT ITEM CONTROL BASED ON SENSOR STATE OR USER PROFILE

(71) Applicant: Adeia Guides Inc., San Jose, CA (US)

(72) Inventors: Charles Dasher, Lawrenceville, GA (US); Christopher Phillips, Hartwell, GA (US)

(21) Appl. No.: 18/216,091

(22) Filed: Jun. 29, 2023

(51) Int. Cl. A61B 5/00; H04N 21/439; H04N 21/488

(52) U.S. Cl. CPC A61B 5/4809; A61B 5/4812; H04N 21/4396; H04N 21/4884

(57) ABSTRACT

Systems and methods for filtering a content item based on a determined sleep state or emotional state of a user are described. A plurality of electronic devices, each having a sensor, or set of sensors, within a predetermined distance of an output device, such as a display device, are detected. Biometric data from the sensors is obtained. The biometric data is associated with a sleep state or emotional state for a user associated with each electronic device. The sleep states vary from being awake to deep sleep. A filter accommodating each determined sleep state is selected and applied to the content item prior to it being displayed on the display device. The content item is displayed on the display device with the applied filter. The filtering process is performed at a processing device or server and not by the display device.

### FIELD OF DISCLOSURE

Embodiments of the present disclosure relate to determining physical or behavioral characteristic(s), such as a sleep state, of one or more individuals based on received sensor inputs and automatically selecting, aggregating, or altering a media content item based on the determined characteristic(s).

### BACKGROUND

Display device manufacturers, such as manufacturers of smart televisions, provide ample settings that can be controlled by a user manually to display and consume a media asset based on their preferred settings. These settings involve manipulating the controls of the smart television to get the desired result. For example, a user who is consuming a media asset on the smart television may change the brightness, volume, contrast, or hue, or activate closed captioning.

Smart televisions may also provide a combination of controls that can be manually selected by the user at one time. For example, the smart television may offer a sport or a cinema mode option to the user. If the user manually selects an option, such as the cinema mode, they may control multiple settings of the smart television to provide a cinematic effect. For example, the selection may turn on surround sound and change settings to mimic a movie theatre.

Although several control options are available, the current systems have several drawbacks and do not address customizing or automating of display of the media asset based on the user's current status. As such, there is a need for a method that overcomes such drawbacks.

### BRIEF DESCRIPTION OF THE DRAWINGS

The various objects and advantages of the disclosure will be apparent upon consideration of the following detailed description, taken in conjunction with the accompanying drawings, in which like reference characters refer to like parts throughout, and in which:

FIG. 1 is a block diagram of a process of detecting a sleep state and applying filters or fetching a content item that is suited to the detected sleep state, in accordance with some embodiments of the disclosure;

FIG. 2 is a block diagram of an example system for detecting a sleep state and applying filters or fetching a content item that is suited for the detected sleep state, in accordance with some embodiments of the disclosure;

FIG. 3 is a block diagram of an electronic device used for detecting a sleep state and transmitting the detected sleep state to a filter module and/or a content item server, in accordance with some embodiments of the disclosure;

FIG. 4 is a block diagram of an example of a system having multiple electronic devices with sensors, a filter module, and a content item server(s) for detecting a sleep state and applying filters or fetching a content item that is suited for the detected sleep state, in accordance with some embodiments of the disclosure;

FIG. 5 is a flowchart of a process for detecting a sleep state and applying filters or fetching a content item that is suited for the detected sleep state, in accordance with some embodiments of the disclosure;

FIG. 6 is a flowchart of a process for detecting a sleep state and applying filters either locally or at server level based on the detected sleep state, in accordance with some embodiments of the disclosure;

FIG. 7 is a block diagram of an example of a sleep state scale used by the system, in accordance with some embodiments of the disclosure;

FIG. 8 depicts an example of a scenario in which the sensor detects a long period of an awake state, in accordance with some embodiments of the disclosure;

FIG. 9 depicts an example of a scenario in which the sensor detects transitioning to a sleep state, in accordance with some embodiments of the disclosure;

FIG. 10 depicts an example of a scenario in which the sensor detects a long period of an asleep state, in accordance with some embodiments of the disclosure;

FIG. 11 depicts an example of a scenario in which the sensor detects transitioning to an awake state or a pending alarm, in accordance with some embodiments of the disclosure;

FIG. 12 depicts an example of a scenario in which the sensor detects long period of an awake state, following an awake transition, in accordance with some embodiments of the disclosure;

FIG. 13 is a flowchart of a process for detecting a sleep state when multiple sensors are present and applying filters based on the rules associated with multiple sensors, in accordance with some embodiments of the disclosure;

FIG. 14 is a block diagram of some examples of sleep state scenarios and types of filters applied in a multiple-sensor environment, in accordance with some embodiments of the disclosure;

FIG. 15 is a block diagram of an example of priority assignment in a multiple-sensor environment, in accordance with some embodiments of the disclosure;

FIG. 16 is a block diagram of an example of inserting a preconfigured video clip into a media stream based on the sleep state detected, in accordance with some embodiments of the disclosure;

FIG. 17 depicts an example of a scenario in which two sensors are detected and each sensor reports a different sleep state, in accordance with some embodiments of the disclosure;

FIG. 18 depicts a conference call in which filters are applied based on a detected sleep state, in accordance with some embodiments of the disclosure;

FIG. 19 depicts an adjustment made to a scheduled alarm based on a detected sleep state, in accordance with some embodiments of the disclosure; and

FIG. 20 is an example of applying a filter applied based on parental control settings, in accordance with some embodiments of the disclosure.

### DETAILED DESCRIPTION

The embodiments disclosed herein help to address the above-mentioned drawbacks and limitations by determining a physical or behavioral characteristic, such as a sleep state, or emotional state of one or more individuals based on received sensor inputs, automatically filtering a media content item based on the determined sleep state by applying a filter that is allocated for the determined characteristic, and displaying or otherwise causing output of the content item on an output device, such as a display or audio device, with the applied filter. By applying such filters, the system dynamically addresses the sleeps states of a user by ensuring that content that is appropriate for a sleep state is outputted or displayed. For example, if a user is sleepy, then the filter applied may reduce the brightness and sound, so the setting is more comfortable for the user. By applying such filters, the system also dynamically addresses conflicts between multiple users. For example, if one user is sleeping and another is watching television, then the filter applied ensures that the television brightness and sound does not disturb the sleeping user while still allowing the user that is consuming the content on the television to continue consuming it. The system may perform various types of dispute resolutions to address sleeps states of multiple users and may also look into their profile setting to determine which filter is most appropriate for a situation when two users have different sleep states, as will be explained in further detail below.

In one embodiment, in a one sensor setting, which is associated with a single user wearing an electronic device having a sensor, biometric data is obtained from the sensor. For example, the biometric data may be obtained from an electronic device such as smart glasses, Wi-Fi earbuds, a smart watch, a smart belt, a heart monitor, an EKG monitor and/or a blood sugar monitor, that are utilized or worn by the user. Although a single sensor incorporated in a single electronic device may be used to implement the techniques disclosed herein, the present disclosure need not be so limited, and a single device that includes one or more sensors, multiple devices worn by the user with each device including one or more sensors, or electronic devices not worn by the user, such as a smart mattress with one or more sensors, an Internet of things (IoT) device that includes one or more sensors and is capable of monitoring one or more users, a smart camera or smart audio device with one or more sensors that is capable of monitoring one or more users, or another type of device, such as a monitoring or sensing device that can obtain biometric readings of one or more users is also contemplated. As such, references to a single sensor incorporated in a single electronic device are merely illustrative of the embodiments, however, any of one or more combinations of devices described herein may also be used.

In some embodiments, methods determine that the device, such as any of one or more of monitoring or sensing devices described above, having the single or multiple sensors is within a predetermined distance from a display or other output device, such as a smart television, smart speaker, or a smart audio/video device. Yet other types of output devices may include devices that are audio only, devices that provide haptic feedback, a variety of IoT devices, such as IoT fan, bed controls, temperature controls, coffee maker, etc. Filters may be applied to any such devices based on sleep state or other emotional or physical states described herein.

The device's distance from the display device may be determined to ensure that the device worn by the user is within a field of view of the display device. In some embodiments, the method may allow the device to be in an adjacent room, as will be further explained below.

The biometric data obtained from the sensor may relate to measuring brain waves, breathing, heart rate, body movement, eye movement, blood sugar oxygen levels, and any other movement or data that can be used to determine a sleep state of a user associated with the electronic device having the sensor. The biometric data may be obtained from the sensor, or a set of sensors, from a device worn by the user or another type of monitoring or sensing device that is not worn by the user but within a predetermined threshold distance of the user. For example, if the biometric data obtains from a smart watch indicates a certain amount of movement, the system may associate such data with the user being awake.

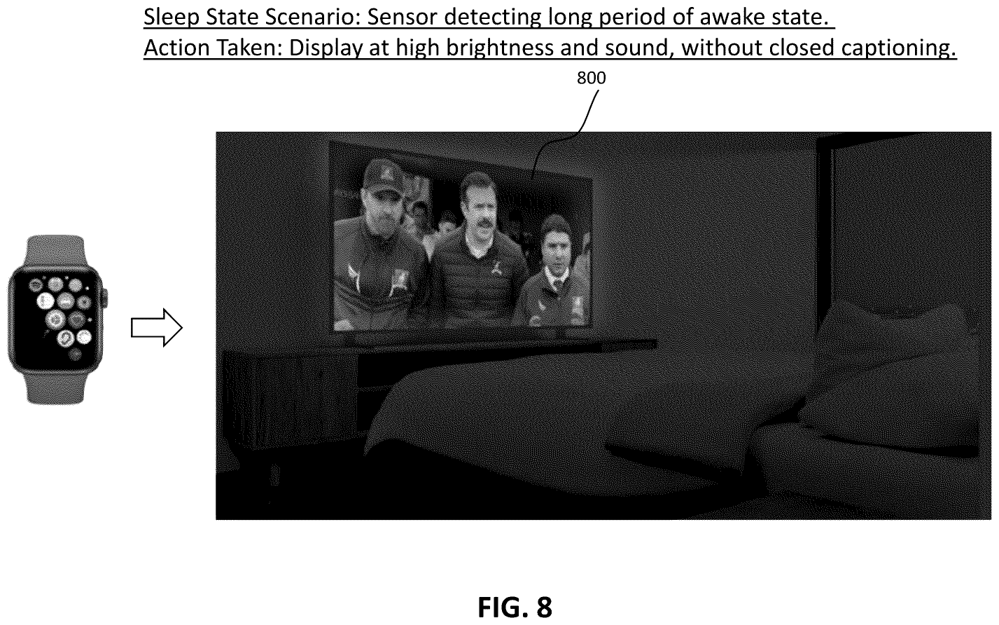

Based on the sleep state determined, a processing device, which may be any device such as a set top box, casting stick, USB stick, streaming stick, or another device capable of applying a filter post decode (i.e., after the media asset or a portion of the media asset has been decoded by a decoder), may be used. Some examples of filters applied are depicted in FIGS. 8-12 and 17-18. The filtering may be performed by a connected processing device and before the content, or a portion of the content, is displayed. The filtering may be applied without the user actively controlling the setting(s) of the display device, such as by providing inputs via controls of the smart television. In other words, without user intervention. The processing device applying the filters allows the flexibility to apply any type of filter, regardless of whether the television has such a display capability. For example, a filter that eliminates a certain sound in the media asset, activates closed captioning, or eliminates blue light, even if such controls are not provided by the display device, may be applied by a connected processing device at the filtering level.

In operation, a media stream that is used to transport a media asset may be received by a decoder of the processing device. In one embodiment, the media asset may be decoded segment by segment by the decoder. A segment that is decoded may be sent to the display device for display or other output device for output. Once a sleep state is detected, a filter may be applied to the next segment at a processing device associated with the display device, once it is decoded but not yet sent to the display device. The type of filter to be applied may be determined based on sleep state data received from the electronic device worn by the user or another type of electronic device that is not worn by the user and can perform monitoring or sensing of the user.

In another embodiment, the media asset may be decoded and immediately after decoding, and before rendering, a filer may be applied. The application of the filter may be performed in real-time in some embodiments. The application of the filter may also be performed in the midst of a segment being decoded such that the system may not need to wait for the completion of the entire segment to be decoded. The type of filter to be applied may be determined based on sleep state data received from the electronic device worn by the user or another type of electronic device that is not worn by the user and can perform monitoring or sensing of the user.

In some embodiments, when the processing device is not capable of applying the filter, the filtering may be performed by a content server. In other embodiments, another server, such as server associated with a third party, ad agency, television program server, may also be used to apply the filter.

The embodiments described also apply in a multiple-sensor environment. In some embodiments, multiple sensors are detected. The multiple sensors are associated with multiple users wearing electronic devices. Biometric data is obtained from each of the multiple sensors and analyzed to determine a sleep state of the user associated with the sensor. For example, the biometric data may be obtained from a first device having a sensor associated with a first user and a second device having a sensor associated with a second user.

The system detecting the plurality of sensors may determine whether the plurality of sensors is within a predetermined distance of a display device. Once it is confirmed that they are within the predetermined distance, biometric data from each of the detected plurality of sensors may be obtained. The biometric data may be correlated with a sleep state for each of the detected plurality of sensors, i.e., for each user associated with the sensor, such as a sleep state for a first user associated with a first device having a sensor.

Upon determining the sleep state, a filter may be selected to be applied to the content that is to be displayed on the display device. The filter selection process may accommodate the sleep state of both the first and the second user. For example, if a sleep state for the first user is awake and the second user is asleep, the filter selected may allow the first user to view the content item without disturbing the second user. This may include turning on closed captioning such that no sound is made. This may also include lowering the brightness such that it is not too bright to wake up the second user, and it may also include adding white noise such that the second user continues to stay asleep. All these filters, and any other filters, that may be applied to the content item are applied by the processing device and before the content, or the portion of the content, is output by the display device. The filtering may be applied without the user controlling the setting of the display device, such as the controls of the smart television.

The process of determining sleep state and selecting a filter may also be applied in an alarm clock setting, conference call setting, or parental control setting. In an alarm clock setting, detecting that a user has gone to bed later than their scheduled time, such as due to consuming a content item, the system may reconfigure or reset the alarm clock, or an alarm placed on another device, by delaying the wake-up time. Such amount of delay time may be the same amount of time the user has delayed going to bed. The delay may allow the user to get the routine amount of sleep the user typically receives if the user had not gone to sleep late.

In one embodiment, in a conference call setting, in response to detecting that a second user who is in the same room as, or next to, the first user is asleep, the system may apply a filter to change the conference call setting such that the conference call doesn't disturb the sleeping second user. Accordingly, the system may apply a filter that turns off conference call video, switches to closed captioning, and uses auto-replies to answer any queries directed at the first user.

In another embodiment, in a conference call setting, in response to detecting that there is external noise or ambient noise, which is noise outside of the conference call itself, the system may apply a filter to block out the external noise or ambient noise such that other participants of the conference call may not hear the external noise or ambient noise that is in the area of one of the participants of the call. For example, user 1 may be on a conference call with user 2. There may be external noise or ambient noise that can be detected by user 1's microphone. Upon detecting the external noise or ambient noise, the system may automatically apply a mute filter to mute user 1's microphone such that the external noise or ambient noise is blocked thereby preventing user 2 from hearing such external noise or ambient noise.

In a parental guidance setting, detecting that a child is either situated in the same room as the adult or that a child has walked in while the adult is consuming content, a parental filter may be applied. In this scenario, both the user's sleep state and the child's presence may be factored in to determine an appropriate filter.

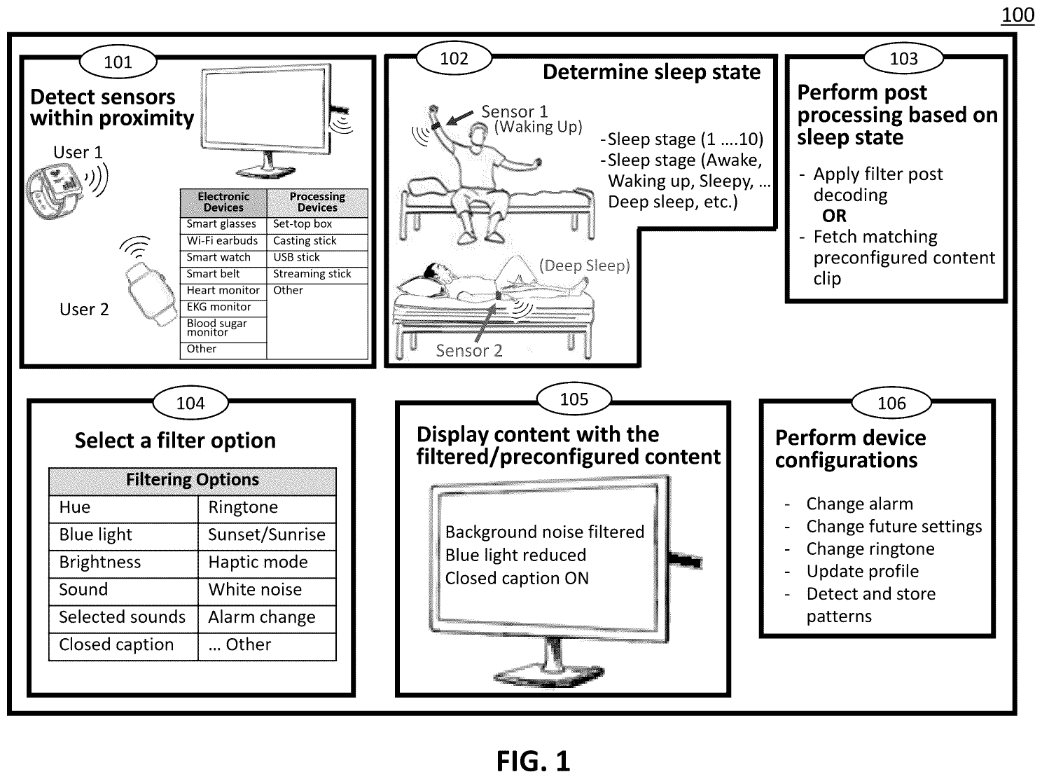

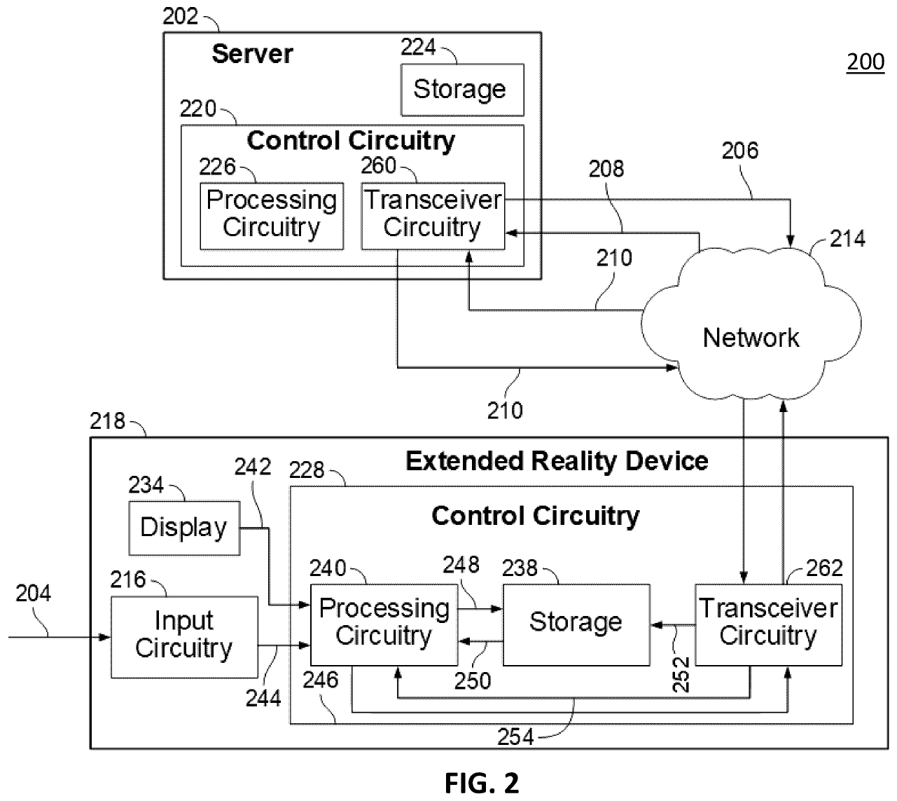

Turning to the figures, FIG. 1 is a block diagram of a process of detecting a sleep state and applying filters or fetching a content item that is suited for the detected sleep state, in accordance with some embodiments of the disclosure. The process 100 may be implemented, in whole or in part, by systems or devices such as those shown in FIGS. 2-4. One or more actions of the process 100 may be incorporated into or combined with one or more actions of any other process or embodiments described herein. The process 100 may be saved to a memory or storage (e.g., any one of those depicted in FIGS. 2-4) as one or more instructions or routines that may be executed by a corresponding device or system to implement the method 100.

In some embodiments, at block 101, the system, such as the system in FIG. 2, via its control circuitry 220 and/or 228, detects a plurality of sensors within a predetermined proximity of the display device.

In some embodiments, the system, via the control circuitry, detects electronic device(s) that each include one or more sensors within the predetermined distance of the display device. As describe above, the references to the single sensor incorporated in the single electronic device are merely for explaining the embodiments, however, any of one or more devices that have one or more sensors that are worn by the user or devices with monitoring and sensing capabilities that are not worn by the user are also contemplated within the embodiments.

The predetermined distance may be determined by the system based on the area surrounding a display device. For example, a bedroom with a display device, such as a TV, may have a total area of between 100 to 200 square feet, with the room dimension being 10′×10′ or 10′×20′ or some other combination to total to between the 100 to 200 square feet area. Based on the size of the bedroom, the system may determine the predetermined distance to be 20 feet, which may be the maximum distance in any one direction in the bedroom, thereby ensuring that any electronic devices that include a sensor that are in the same bedroom as the display device are detected. The predetermined distance may also be a standard number that is used for all areas. The predetermined distance may also be adjusted by the system or changed by a user associated with the system.

In other embodiments, the predetermined distance may include a distance beyond a current room or confined space. For example, in a multiple-sensor scenario, a first electronic device having a sensor may be located in the same room and a second electronic device having a second sensor may be located in an adjacent room. As will be further described in the multiple-electronic devices/sensors scenarios below, the predetermined distance may include, in some embodiments, a distance that covers multiple adjacent rooms such that, for example, a TV in a first room used by a user wearing the first electronic device is not loud enough to disturb a second user who is wearing a second electronic device with a sensor, is in an adjacent room, and is currently asleep.

In some embodiments, the system may access data, such as biometric and other sensor data, from a user wearing an electronic device to determine their sleep state. This may be data obtained from devices worn by the user, such as smart glasses, Wi-Fi earbuds, a smart watch, a smart belt, a heart monitor, an EKG monitor, a blood sugar monitor, etc. Any other electronic device that includes a sensor, or body-worn sensors, is also contemplated for obtaining a sleep state of a user.

In some embodiments, the user may be wearing one or more electronic devices on their body. For example, the user may be wearing smart glasses, Wi-Fi earbuds, smart watch, smart belt, heart monitor, EKG monitor, blood sugar monitor or some other type of electronic device or a sensor through which biomarker input, such as their sleep state, can be obtained.

In some embodiments, the user may also be wearing motion sensors or trackers that monitor the user's movements and can be used to deduce the user's sleep state. For example, the user may rotate their hand that is wearing a ring with a sensor in it that can provide movement data relating to the user's movement of their hand. Such data may be obtained to determine the user's sleep state.

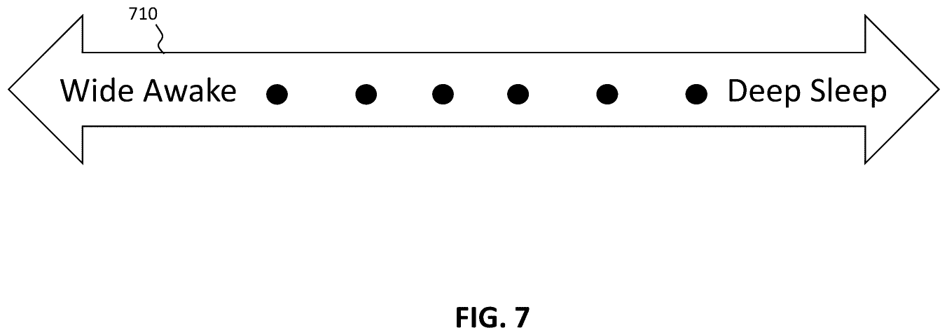

In one embodiment, the electronic device may be a pair of smart glasses worn by the user. The current user may be wearing these smart glasses while within the predetermined distance of the display device. The control circuitry 220 and/or 228 may access a part of the smart glasses, such as its inward-facing camera, to obtain the user's sleep state. For example, the control circuitry 220 and/or 228 may obtain the user's gaze, dilation in the user's eyes, eye pigmentation (which can indicate sleepiness) as compared to a baseline pigmentation obtained earlier, degree of attention, frequency of eyes closing, or water content in the eyes as an indication of their sleep state. For example, in a scenario where the user is very tired and sleepy, their eyes may have water built up or they may frequently shut their eyelids. Such data may be used to determine whether the user is awake, asleep, or in various degrees of awareness and sleepiness, as depicted in one example of a sleep state scale in FIG. 7.

In one embodiment, the electronic device may be a pair of earbuds with sensors worn by the user. In some embodiments, the earbuds may include an encephalography sensor that may be used for monitoring sleep. These sensors may be similar to electroencephalogram (EEG) sensors but worn in the ear. Some existing systems that have ear-EEG sensors include memory-foam viscoelastic earpieces with electrodes that are positioned in diametrically opposed locations and are made from flexible conductive cloth. The control circuitry 220 and/or 228 may access the ear-EEG sensors to obtain the user's sleep state. For example, the ear-EEG sensor may detect activity, including brain activity, when combined with another sensor placed on the user's head, to determine the user's sleep state. The data obtained may be used to determine whether the user is awake, asleep, or in various degrees of awareness and sleepiness as depicted in one example of a sleep state scale in FIG. 7.

In one embodiment, the user may be wearing a smart watch. The user may be wearing the smart watch while within the predetermined distance from the display device. The control circuitry 220 and/or 228 may access the smart watch to obtain any biometric readings from it. Some smart watches include several sensors, such as 16+ sensors. These sensors may be fitness trackers, movement trackers, altimeters, optical heart rate sensors, blood sugar oxygen sensors, bioimpedance sensors, EKG sensors, gyroscopes, GPS sensors, electrodermal activity sensors, skin temperature sensors and more. The control circuitry 220 and/or 228 may access the sensors to obtain data, such as biometric and other sensor data, that may serve as an indicating factor of the user's sleep state. For example, the control circuitry 220 and/or 228 may perform actigraphy to determine sleep state. Actigraphy, which is a method to detect movement or heart rate, allows the smart watch to detect the user's movement, which can be tracked to determine the sleep state as well as sleep patterns. For example, if a user is tossing and turning or moving a lot while in bed, the movement detection may be used to determine that the user is not in deep sleep or is uncomfortable and in a light sleep mode.

In one embodiment, the user may be wearing a smart belt. The user may be wearing the smart belt while within the predetermined distance of the display device. Although a smart belt, which includes a sensor, is described, any type of smart clothing or wearable that includes a sensor is also contemplated in the embodiments. The control circuitry 220 and/or 228 may access the smart belt to obtain biomarker data and movements from it. For example, the control circuitry 220 and/or 228 may determine that a user wearing the smart belt is tossing and turning. The control circuitry 220 and/or 228 may also determine that there is no weight or pressure applied on the smart belt which is indicative of the user sitting up. For example, if the user wearing the smart belt were lying in bed, their body weight would press the smart belt against the bed. The data obtained, such as biometric and other sensor data, may be used to determine whether the user is awake, asleep, or in various degrees of awareness and sleepiness as depicted in one example of a sleep state scale in FIG. 7.

In one embodiment, the user may be wearing a heart rate monitor. The user may be wearing such a heart rate monitor while within the predetermined distance from the display device. The control circuitry 220 and/or 228 may access the heart rate monitor to obtain a heart rate from it, and heart or pulse rate may be correlated with the user's sleep state. Since heart rate is slower when a user is sleeping as compared to when the user is awake or performing an activity, even if the activity is simply sitting in bed, such heart rate data obtained may be used to determine whether the user is awake, asleep, or in various degrees of awareness and sleepiness as depicted in one example of a sleep state scale in FIG. 7.

The heart rate data obtained may also allow the control circuitry 220 and/or 228 to detect different stages of sleep and transition from one stage to another. For example, the control circuitry 220 and/or 228 may track the increase or decrease of heart rate over time and correlate that with a different sleep state. The heart rate slowing to its slowest from a moderate heart rate, for example, may be indicative of the user transitioning from sleep to deep sleep or REM sleep state, which has the lowest heart rate.

In one embodiment, the user may be wearing a device with a pressure sensor, or an EKG reader. The data, such as biometric and other sensor data, obtained from such devices may be used to determine whether the user is awake, asleep, or in various degrees of awareness and sleepiness as depicted in one example of a sleep state scale in FIG. 7.

In one embodiment, the user may be wearing a device that monitors blood sugar level. The control circuitry 220 and/or 228 may access such a device and detect the current blood sugar level of the user. Since blood sugar levels may increase as part of the heart's rhythmic cycle when a user is sleeping, the data obtained from such blood sugar level measuring devices may be used to determine whether the user is awake, asleep, or in various degrees of awareness and sleepiness as depicted in one example of a sleep state scale in FIG. 7.

Although a few examples of electronic devices that include sensors have been described above, the embodiments are not so limited. Any other type of electronic device, sensor, or equipment that can measure brain waves, breathing, heart rate, body movement, eye movement, blood sugar oxygen levels, and any other movement or data that can be used to determine a sleep state is also contemplated in the embodiments. Devices, sensors, and equipment that measure motion while in bed are also contemplated in the embodiments. The data from such motion measurement and detection devices, sensors, and equipment can be used to determine sleep state of the user. For example, if a certain amount of movement has occurred, the system may associate an allowed level of movement with being awake, and periods of being still with the user being asleep.

Although some embodiments of electronic devices that can be worn by the user are described above, other types of electronic devices, sensors, or equipment that are not worn by the user are also contemplated within the embodiments.

One embodiment of such an equipment that is not worn by the user is a smart mattress. This may be a mattress that include one or more sensors on which a user may lay down. The mattress, based on one or more sensors embedded in the mattress, may detect movement of a user laying down upon the mattress. Such data may be transmitted to the control circuitry 220 and/or 228 to determine the sleep state of the user laying upon the mattress. In some embodiments, the sensor(s) in the mattress may determine which user is laying down upon the mattress by detecting wearable devices worn by the user. For example, the sensors in the mattress may communicate with the one or more sensors in a user's wearable device to confirm the identity of the user.

Another embodiment of a non-wearable device used to determine the sleep state may be a security camera that is located in the same room as the user. In this embodiment, the security camera may monitor the user(s) in the room and transmit the activity data to the control circuitry 220 and/or 228 to determine the sleep state of the user. If the security camera footage indicates that the user's eyes are open, then the control circuitry 220 and/or 228, such as by using an AI algorithm, may determine that the user's sleep state is awake.

In yet another embodiment, the non-wearable device may be a mobile phone. Its eye-gazing and eye-tracking technology may be used to determine the sleep state of a user. For example, the inward facing camera may detect the user's eye blink rate, sleepy or tired eyes, or wide-awake eyes. It may also detect distraction when the user is looking elsewhere and not at a media display or output device. Such data may be transmitted to the control circuitry 220 and/or 228 to determine the sleep state of the user.

Motion detectors, sound detectors, and other sensing devices that are not worn by the user may also be used to monitor the user and transmit monitored data to the control circuitry 220 and/or 228 to determine the sleep state of the user.

In addition to applying a filter based on a sleep state, other distractions, disturbances, or things that may not be pleasant to the user, such as a loud sound in the media asset, or a scary sound to an animal, may be detected and used in determining which filter to apply.

At block 101, as depicted, in one embodiment, there may be two electronic devices that have sensors and are worn by User 1 and User 2 detected by the control circuitry 220 and/or 228. Although two electronic devices that have sensors are depicted, the embodiments are not so limited, and the process 100 may be applied to a single or any number of multiple electronic devices.

The processing device, which may be any device such as a set-top box, casting stick, USB stick, streaming stick, or another device capable of applying a filter, post decoding of the media asset or a portion of the media asset stage, may be used. Some examples of current processing devices that can be used to add the unique filtering embodiments discussed herein based on sleep state include TiVo Edge™, Chromecast™ with Google TV™, Roku™ streaming stick, FireTV™ stick, Anycast™ streaming stick, Apple TV™, Nvidia Shield™, and Amazon Fire TV Cube™.

In some embodiments, the processing device may continuously monitor for an electronic device within a predetermined distance. It may perform the monitoring by detecting Bluetooth signals, Wi-Fi connections, or by using a camera associated with the display device to determine if any user wearing an electronic device has entered a room or is within a predetermined distance of the display device.

The monitoring may be triggered by the processing device at all times, at predetermined intervals, or whenever an electronic device establishes a Bluetooth or Wi-Fi connection.

At block 102, in some embodiments, the control circuitry 220 and/or 228 determines a sleep state of User 1, who may be wearing an electronic device with Sensor 1 and a sleep state of User 2, who may be wearing an electronic device with Sensor 2. The electronic devices may be any of the electronic devices described in block 101.

The sleep states may be determined by the control circuitry 220 and/or 228 by accessing the electronic devices worn by User 1 and 2. The control circuitry 220 and/or 228 may access data from sensors in the electronic device that provide readings relating to the user's brain waves, breathing, heart rate, body movement, eye movement, blood sugar oxygen levels, and any other movement or data that can be used to determine a sleep state. The control circuitry 220 and/or 228 may process the readings obtained and correlate them with a sleep state for each user. For example, a slower heart rate reading or a reading that shows lack of motion or no movement or still condition for a certain period of time may be associated with the user being asleep, while higher heart rate readings or movement above a threshold may be associated with an awake state.

The sleep state may be quantified in a variety of scales. In some embodiments, the sleep scale may range from 1-10, 1-100, A-Z or some other numbering system. In other embodiments, as depicted in FIG. 7, the sleep scale may be a range from wide awake to deep sleep. There may be several degrees and ranges of sleep states within the scales described herein. There may also be several substages, which are transition stages between each sleep state.

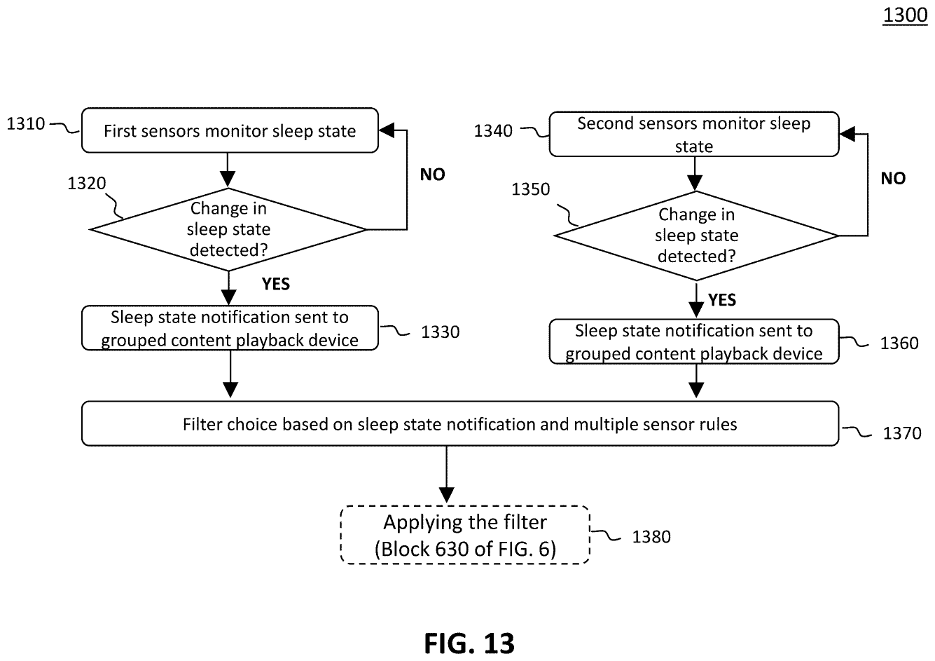

In some embodiments, as depicted at block 102, the sleep state of User 1 wearing an electronic device with Sensor 1 was determined to be waking up, and the sleep state of User 2 wearing an electronic device with Sensor 2 was determined to be deep sleep. It may also be determined by the control circuitry 220 and/or 228 that both User 1 and User 2 are within the predetermined distance of the processing device or the display device of FIG. 1. This may mean that User 1 and User 2 may be in the same bed next to each other or may be in separate beds that are in the same room. Further details relating to the process for determining sleep states of multiple users wearing electronic devices with sensors are described in the description related to FIG. 13.

At block 103, once the sleep states of Users 1 and 2 are determined, the control circuitry 220 and/or 228 may perform post processing of a media asset based on the determined sleep states.

In some embodiments, the post processing based on sleep state may be applying a filter post decoding. Some examples of filters applied are depicted in FIGS. 8-12 and 17-18. In some embodiments, the control circuitry 220 and/or 228 use the processing device to apply the filter based on the determined sleep state. This would mean that the media asset is filtered at the processing device before it is displayed on the display device, such as a TV, that is connected to the processing device. For example, if a transition from an awake state to a sleep state is determined, then a filter that lowers the volume and brightness may be applied at the processing device such that, once it is applied, the resulting display of the media asset on the display device is displayed with a lower volume and brightness.

The application of filters discussed herein are applied by the control circuitry 220 and/or 228 at the processing device and not at the display device. In other words, the brightness of the television or the volume of the television is not lowered by adjusting the television volume and brightness controls. Instead, the filters are being applied at the post decoding stage at the processing device. Applying the filters at the processing device allows the control circuitry 220 and/or 228 to apply any type of filter configured at the processing device regardless of whether the television has such a display capability. For example, a filter that eliminates a certain sound in the media asset, activates closed captioning, and eliminates blue light in the media asset may be determined to be the best filter for the sleep state determined. Such may be all encompassed in one filter or separate filters. In this example, if the television does not have such a capability to perform those operations, e.g., eliminate blue light, etc., which may be due to the television being an older model, the control circuitry 220 and/or 228 may still accomplish the end result by applying the associated filter at the processing device to cause the media asset to be displayed in the desired manner, e.g., with the blue light eliminated.

In operation, a media stream that is used to transport a media asset may be received by a decoder of the processing device. The media asset may be decoded segment by segment by the decoder. The segment that is decoded may be sent to the display device for display. Once a sleep state is detected, a filter may be applied to the next segment at a processing device associated with the display device, once it is decoded but not yet sent to the display device. The type of filter to be applied may be determined based on sleep state data received from the electronic device worn by the user.

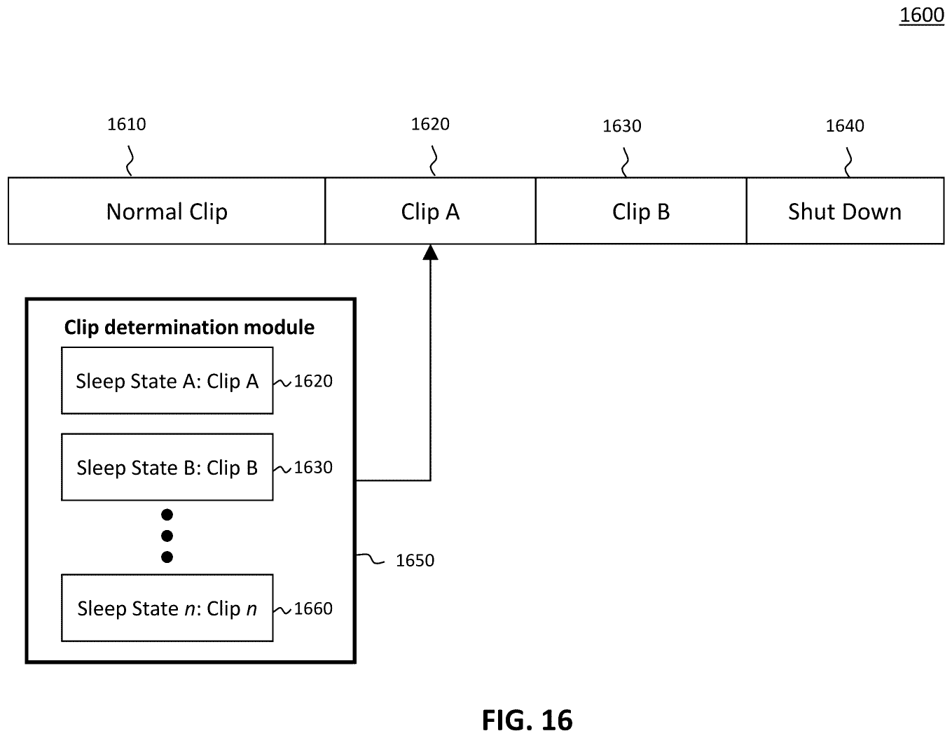

In another embodiment, the media asset may be decoded and immediately after decoding, and before rendering, a filer may be applied. The application of the filter may be performed in real-time in some embodiments. The application of the filter may also be performed in the midst of a segment being decoded such that the system may not need to wait for the completion of the entire segment to be decoded. The type of filter to be applied may be determined based on sleep state data received from the electronic device worn by the user or another type of electronic device that is not worn by the user and can perform monitoring or sensing of the user. The filter may be applied at the processing device or when the processing device is not capable at the content server. In some embodiments, the post processing based on sleep state may be fetching a preconfigured content item clip and displaying the fetched content item clip or segment. Further details relating to fetching clips/segments and inserting them in the media stream are described in the description related to FIG. 16.

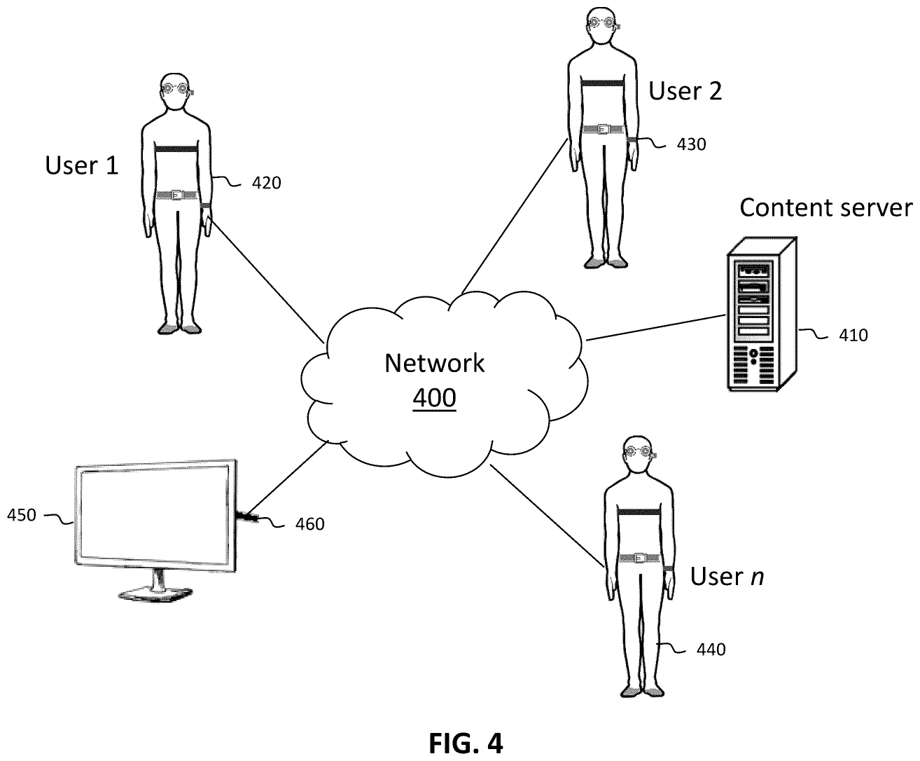

In this embodiment, the media stream that is used to transport a media asset may be received by a decoder of the processing device. A portion of the media asset received may be decoded by the decoder. The portion/segment that is decoded may be sent to the display device for display or an output device, such as an audio device, for output. Once a sleep state is detected, the processing device may signal to a content item server, such as the content item server 410 of FIG. 4, to provide the next portion/segment/clip that is associated with the sleep state detected. In this embodiment, a plurality of preconfigured portions/segments/clips for each sleep state may be premade. Once a sleep state is determined, a matching process may be performed to find a segment of the media asset that is premade for the determined sleep state. If such a version exists, then it may be injected into the media stream, received by the processing device, and then sent to the display device for display. If such a version that matches the sleep state does not exist, then the current media stream may be played as is with no changes, or a filter may be applied at the processing device as described above.

In another embodiment, the sleep state may be defined in the manifest file. In this embodiment, based on the detected sleep state, the processing device may request an encoded segment matching the calculated bit rate to the display device within the choice of media asset segments matching the current sleep state.

At block 104, a filter to be applied may be selected. The selection may be based on the sleep state detected. Some examples of filters include, adding, removing, increasing, decreasing, or making other changes to hue, blue light, brightness, sound, selection of sounds, closed captioning, ringtone, sunset/sunrise, haptic mode, white noise, and alarm. These may be some of the filtering options that can be applied, and their degree of application may depend on the sleep state. Although some examples of filters are depicted at block 104, the embodiments are not so limited. Additional types of filters are also contemplated in the embodiments.

At block 105, once the filter is applied at the processing device, the media segment with the filter applied may be rendered on the display device. For example, if a user is watching a movie and has consumed the first 33 minutes, once a sleep state is determined, the portion of the movie that sequentially follows the 33-minute mark may be displayed with the filter applied. The control circuitry 220 and/or 228 may continue to track changes in sleep state and continue to apply additional filters or adjust the degree of the applied filter to match the changing sleep state.

At block 106, the control circuitry 220 and/or 228 may perform device configurations as needed. In some embodiments, the device configurations that can be applied include changing the time of an upcoming alarm, changing future settings of the device such as all subsequent alarms, changing the ringtone, updating the user profile based on sleep states determined, and detecting and storing sleep patterns. Although some examples of device configuration filters are depicted at block 106, the embodiments are not so limited. Additional types of device configurations are also contemplated in the embodiments.

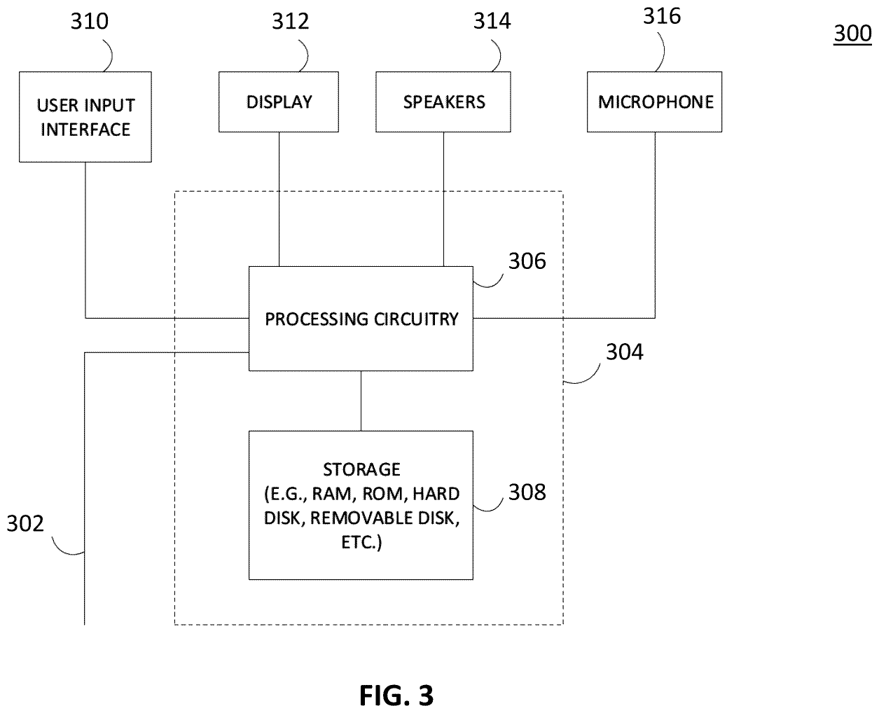

FIG. 2 is a block diagram of an example system for detecting a sleep state and applying filters or fetching a content item that is suited for the detected sleep state, in accordance with some embodiments of the disclosure and FIG. 3 is a block diagram of an electronic device used for detecting a sleep state and transmitting the detected sleep state to a filter module and/or a content item server, in accordance with some embodiments of the disclosure.

FIGS. 2 and 3 also describe example devices, systems, servers, and related hardware that may be used to implement processes, functions, and functionalities described at least in relation to FIGS. 1, 5-20. Further, FIGS. 2 and 3 may also be used for detecting sensors and electronic devices with sensors within proximity of a display device, determining the type of sensor, such as whether it is embedded in a smart glasses, smart watch, EKG monitor, blood sugar monitor etc., obtaining sleep stage associated with a user wearing the electronic device with the sensor, when multiple users are present, then obtaining sleep stage associated with multiple users wearing the electronic device with the sensor, applying filters to media asset or a portion/segment of the media asset based on a determination of sleep state of a user(s) after it has been decoded, fetching preconfigured content for a particular sleep state when available, selecting one or more filters, performing alarm and device configurations, accessing user profiles, detecting and storing user behavior patterns as it relates to previous filters applied, changing ringtones, determining conference call setting and filters to be applied during a conference call if a second user adjacent to the first user is asleep, automatically applying device configurations, detecting changes in a sleep state and, performing functions related to all other processes and features described herein.

In some embodiments, one or more parts of, or the entirety of system 200, may be configured as a system implementing various features, processes, functionalities and components of FIGS. 1, and 5-20. Although FIG. 2 shows a certain number of components, in various examples, system 200 may include fewer than the illustrated number of components and/or multiples of one or more of the illustrated number of components.

System 200 is shown to include a computing device 218, a server 202 and a communication network 214. It is understood that while a single instance of a component may be shown and described relative to FIG. 2, additional instances of the component may be employed. For example, server 202 may include, or may be incorporated in, more than one server. Similarly, communication network 214 may include, or may be incorporated in, more than one communication network. Server 202 is shown communicatively coupled to computing device 218 through communication network 214. While not shown in FIG. 2, server 202 may be directly communicatively coupled to computing device 218, for example, in a system absent or bypassing communication network 214.

Communication network 214 may comprise one or more network systems, such as, without limitation, an internet, LAN, WIFI or other network systems suitable for audio processing applications. In some embodiments, system 200 excludes server 202, and functionality that would otherwise be implemented by server 202 is instead implemented by other components of system 200, such as one or more components of communication network 214. In still other embodiments, server 202 works in conjunction with one or more components of communication network 214 to implement certain functionality described herein in a distributed or cooperative manner. Similarly, in some embodiments, system 200 excludes computing device 218, and functionality that would otherwise be implemented by computing device 218 is instead implemented by other components of system 200, such as one or more components of communication network 214 or server 202 or a combination. In still other embodiments, computing device 218 works in conjunction with one or more components of communication network 214 or server 202 to implement certain functionality described herein in a distributed or cooperative manner.

Computing device 218 includes control circuitry 228, display 234 and input circuitry 216. Control circuitry 228 in turn includes transceiver circuitry 262, storage 238 and processing circuitry 240. In some embodiments, computing device 218 or control circuitry 228 may be configured as electronic device 300 of FIG. 3.

Server 202 includes control circuitry 220 and storage 224. Each of storages 224 and 238 may be an electronic storage device. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 4D disc recorders, digital video recorders (DVRs, sometimes called personal video recorders, or PVRs), solid state devices, quantum storage devices, gaming consoles, gaming media, or any other suitable fixed or removable storage devices, and/or any combination of the same. Each storage 224, 238 may be used to store various types of content (e.g., distances of user electronic devices that include sensors from the display device, sleep states, sleep state user preferences, user profiles, filtering options, decoded segments of a media asset, filtered segments of a media asset, device configurations and changes to the configurations, data relating to device alarms, sleep timings of a user, conference call preferences when certain sleep states are detected, sensor data that can be associated with a sleep state). Non-volatile memory may also be used (e.g., to launch a boot-up routine, launch an app, render an app, and other instructions). Cloud-based storage may be used to supplement storages 224, 238 or instead of storages 224, 238. In some embodiments, data relating to distances of user electronic devices that include sensors from the display device, sleep states, sleep state user preferences, user profiles, filtering options, decoded segments of a media asset, filtered segments of a media asset, device configurations and changes to the configurations, data relating to device alarms, sleep timings of a user, conference call preferences when certain sleep states are detected, sensor data that can be associated with a sleep state, and data relating to all other processes and features described herein, may be recorded and stored in one or more of storages 212, 238.

In some embodiments, control circuitries 220 and/or 228 executes instructions for an application stored in memory (e.g., storage 224 and/or storage 238). Specifically, control circuitries 220 and/or 228 may be instructed by the application to perform the functions discussed herein. In some implementations, any action performed by control circuitries 220 and/or 228 may be based on instructions received from the application. For example, the application may be implemented as software or a set of executable instructions that may be stored in storage 224 and/or 238 and executed by control circuitries 220 and/or 228. In some embodiments, the application may be a client/server application where only a client application resides on computing device 218, and a server application resides on server 202.

The application may be implemented using any suitable architecture. For example, it may be a stand-alone application wholly implemented on computing device 218. In such an approach, instructions for the application are stored locally (e.g., in storage 238), and data for use by the application is downloaded on a periodic basis (e.g., from an out-of-band feed, from an internet resource, or using another suitable approach). Control circuitry 228 may retrieve instructions for the application from storage 238 and process the instructions to perform the functionality described herein. Based on the processed instructions, control circuitry 228 may determine a type of action to perform in response to input received from input circuitry 216 or from communication network 214. For example, in response to determining a sleep state of a user, the control circuitry 228 may determine a filter that is suitable, preferred, or allocated for the determined for the determined sleep state and apply the filter to the decoded media asset or segment of the media asset. It may also perform steps of processes described in FIGS. 1, 5, 6, 13, and 16.

In client/server-based embodiments, control circuitry 228 may include communication circuitry suitable for communicating with an application server (e.g., server 202) or other networks or servers. The instructions for carrying out the functionality described herein may be stored on the application server. Communication circuitry may include a cable modem, an Ethernet card, or a wireless modem for communication with other equipment, or any other suitable communication circuitry. Such communication may involve the internet or any other suitable communication networks or paths (e.g., communication network 214). In another example of a client/server-based application, control circuitry 228 runs a web browser that interprets web pages provided by a remote server (e.g., server 202). For example, the remote server may store the instructions for the application in a storage device. The remote server may process the stored instructions using circuitry (e.g., control circuitry 228) and/or generate displays. Computing device 218 may receive the displays generated by the remote server and may display the content of the displays locally via display 234. This way, the processing of the instructions is performed remotely (e.g., by server 202) while the resulting displays, such as the display windows described elsewhere herein, are provided locally on computing device 218. Computing device 218 may receive inputs from the user via input circuitry 216 and transmit those inputs to the remote server for processing and generating the corresponding displays. Alternatively, computing device 218 may receive inputs from the user via input circuitry 216 and process and display the received inputs locally, by control circuitry 228 and display 234, respectively.

Server 202 and computing device 218 may transmit and receive content and data distances of user electronic devices that include sensors from the display or another type of output device, sleep states, sleep state user preferences, user profiles, filtering options, decoded segments of a media asset, filtered segments of a media asset, device configurations and changes to the configurations, data relating to device alarms, sleep timings of a user, conference call preferences when certain sleep states are detected, ambient and surrounding noise preferences during the conference call, such a muting microphone or enabling closed captions, and other sensor data that can be associated with a sleep state. Control circuitry 220, 228 may send and receive commands, requests, and other suitable data through communication network 214 using transceiver circuitry 260, 262, respectively. Control circuitry 220, 228 may communicate directly with each other using transceiver circuits 260, 262, respectively, avoiding communication network 214.

It is understood that computing device 218 is not limited to the embodiments and methods shown and described herein. In nonlimiting examples, computing device 218 may be an electronic device, a personal computer (PC), a laptop computer, a tablet computer, a WebTV box, a personal computer television (PC/TV), a PC media server, a PC media center, a handheld computer, a mobile telephone, a smartphone, a virtual, augmented, or mixed reality device, or a device that can perform function in the metaverse, or any other device, computing equipment, or wireless device, and/or combination of the same capable of suitably determining sleep state of a user, changes in the sleep state, and applying a filter that is associated with the determined sleep state, and displaying the content with the filter applied on the display device.

Control circuitries 220 and/or 218 may be based on any suitable processing circuitry such as processing circuitry 226 and/or 240, respectively. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores). In some embodiments, processing circuitry may be distributed across multiple separate processors, for example, multiple of the same type of processors (e.g., two Intel Core i9 processors) or multiple different processors (e.g., an Intel Core i7 processor and an Intel Core i9 processor). In some embodiments, control circuitries 220 and/or control circuitry 218 are configured for detecting sensors and electronic devices with sensors within proximity of a display device, determining the type of sensor, such as whether it is embedded in a smart glasses, smart watch, EKG monitor, blood sugar monitor etc., obtaining sleep stage associated with a user wearing the electronic device with the sensor, when multiple users are present, then obtaining sleep stage associated with multiple users wearing the electronic device with the sensor, applying filters to media asset or a portion/segment of the media asset based on a determination of sleep state of a user(s) after it has been decoded, fetching preconfigured content for a particular sleep state when available, selecting one or more filters, performing alarm and device configurations, accessing user profiles, detecting and storing user behavior patterns as it relates to previous filters applied, changing ringtones, determining conference call setting and filters to be applied during a conference call if a second user adjacent to the first user is asleep, automatically applying device configurations, detecting changes in a sleep state and, and performing functions related to all other processes and features described herein.

Computing device 218 receives a user input 204 at input circuitry 216. For example, computing device 218 may receive data relating to distances of user electronic devices that include sensors from the display device, sleep states, sleep state user preferences, user profiles, filtering options, decoded segments of a media asset, filtered segments of a media asset, device configurations and changes to the configurations, data relating to device alarms, sleep timings of a user, conference call preferences when certain sleep states are detected, sensor data that can be associated with a sleep state.

Transmission of user input 204 to computing device 218 may be accomplished using a wired connection, such as an audio cable, USB cable, ethernet cable or the like attached to a corresponding input port at a local device, or may be accomplished using a wireless connection, such as Bluetooth, WIFI, WiMAX, GSM, UTMS, CDMA, TDMA, 3G, 4G, 4G LTE, or any other suitable wireless transmission protocol. Input circuitry 216 may comprise a physical input port such as a 3.5 mm audio jack, RCA audio jack, USB port, ethernet port, or any other suitable connection for receiving audio over a wired connection or may comprise a wireless receiver configured to receive data via Bluetooth, WIFI, WiMAX, GSM, UTMS, CDMA, TDMA, 3G, 4G, 4G LTE, or other wireless transmission protocols.

Processing circuitry 240 may receive input 204 from input circuit 216. Processing circuitry 240 may convert or translate the received user input 204 that may be in the form of voice input into a microphone, or movement or gestures to digital signals. In some embodiments, input circuit 216 performs the translation to digital signals. In some embodiments, processing circuitry 240 (or processing circuitry 226, as the case may be) carries out disclosed processes and methods. For example, processing circuitry 240 or processing circuitry 226 may perform processes as described in FIGS. 1, 5, 6, 13, and 16, respectively.

FIG. 3 is a block diagram of an electronic device used for detecting a sleep state and transmitting the detected sleep state to a filter module and/or a content item server, in accordance with some embodiments of the disclosure. In an embodiment, the equipment device 300, is the same equipment device 202 of FIG. 2. The equipment device 300 may receive content and data via input/output (I/O) path 302. The I/O path 302 may provide audio content (e.g., such as in the speakers of an XR headset). The control circuitry 304 may be used to send and receive commands, requests, and other suitable data using the I/O path 302. The I/O path 302 may connect the control circuitry 304 (and specifically the processing circuitry 306) to one or more communications paths or links (e.g., via a network interface), any one or more of which may be wired or wireless in nature. Messages and information described herein as being received by the equipment device 300 may be received via such wired or wireless communication paths. I/O functions may be provided by one or more of these communications paths or intermediary nodes but are shown as a single path in FIG. 3 to avoid overcomplicating the drawing.

The control circuitry 304 may be based on any suitable processing circuitry such as the processing circuitry 306. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 or i9 processor).

In client-server-based embodiments, the control circuitry 304 may include communications circuitry suitable for detecting sensors and electronic devices with sensors within proximity of a display device, determining the type of sensor, such as whether it is embedded in a smart glasses, smart watch, EKG monitor, blood sugar monitor etc., obtaining sleep stage associated with a user wearing the electronic device with the sensor, when multiple users are present, then obtaining sleep stage associated with multiple users wearing the electronic device with the sensor, applying filters to media asset or a portion/segment of the media asset based on a determination of sleep state of a user(s) after it has been decoded, fetching preconfigured content for a particular sleep state when available, selecting one or more filters, performing alarm and device configurations, accessing user profiles, detecting and storing user behavior patterns as it relates to previous filters applied, changing ringtones, determining conference call setting and filters to be applied during a conference call if a second user adjacent to the first user is asleep, automatically applying device configurations, detecting changes in a sleep state and, and performing functions related to all other processes and features described herein.

The instructions for carrying out the above-mentioned functionality may be stored on one or more servers. Communications circuitry may include a cable modem, an integrated service digital network (ISDN) modem, a digital subscriber line (DSL) modem, a telephone modem, ethernet card, or a wireless modem for communications with other equipment, or any other suitable communications circuitry. Such communications may involve the internet or any other suitable communications networks or paths. In addition, communications circuitry may include circuitry that enables peer-to-peer communication of primary equipment devices, or communication of primary equipment devices in locations remote from each other (described in more detail below).

Memory may be an electronic storage device provided as the storage 308 that is part of the control circuitry 304. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid-state devices, quantum-storage devices, gaming consoles, gaming media, or any other suitable fixed or removable storage devices, and/or any combination of the same. The storage 308 may be used to store various types of content, (e.g., distances of user electronic devices that include sensors from the display device, sleep states, sleep state user preferences, user profiles, filtering options, decoded segments of a media asset, filtered segments of a media asset, device configurations and changes to the configurations, data relating to device alarms, sleep timings of a user, conference call preferences when certain sleep states are detected, ambient and surrounding noise preferences during the conference call, such a muting microphone or enabling closed captions, and other sensor data that can be associated with a sleep state). Cloud-based storage, described in relation to FIG. 3, may be used to supplement the storage 308 or instead of the storage 308.

The control circuitry 304 may include audio generating circuitry and tuning circuitry, such as one or more analog tuners, audio generation circuitry, filters or any other suitable tuning or audio circuits or combinations of such circuits. The control circuitry 304 may also include scaler circuitry for upconverting and down converting content into the preferred output format of the electronic device 300. The control circuitry 304 may also include digital-to-analog converter circuitry and analog-to-digital converter circuitry for converting between digital and analog signals. The tuning and encoding circuitry may be used by the electronic device 300 to receive and to display, to play, or to record content. The circuitry described herein, including, for example, the tuning, audio generating, encoding, decoding, encrypting, decrypting, scaler, and analog/digital circuitry, may be implemented using software running on one or more general purpose or specialized processors. If the storage 308 is provided as a separate device from the electronic device 300, the tuning and encoding circuitry (including multiple tuners) may be associated with the storage 308.

The user may utter instructions to the control circuitry 304, which are received by the microphone 316. The microphone 316 may be any microphone (or microphones) capable of detecting human speech. The microphone 316 is connected to the processing circuitry 306 to transmit detected voice commands and other speech thereto for processing. In some embodiments, voice assistants (e.g., Siri, Alexa, Google Home and similar such voice assistants) receive and process the voice commands and other speech.

The electronic device 300 may include an interface 310. The interface 310 may be any suitable user interface, such as a remote control, mouse, trackball, keypad, keyboard, touchscreen, touchpad, stylus input, joystick, or other user input interfaces. A display 312 may be provided as a stand-alone device or integrated with other elements of the electronic device 300. For example, the display 312 may be a touchscreen or touch-sensitive display. In such circumstances, the interface 310 may be integrated with or combined with the microphone 316. When the interface 310 is configured with a screen, such a screen may be one or more monitors, a television, a liquid crystal display (LCD) for a mobile device, active-matrix display, cathode-ray tube display, light-emitting diode display, organic light-emitting diode display, quantum-dot display, or any other suitable equipment for displaying visual images. In some embodiments, the interface 310 may be HDTV-capable. In some embodiments, the display 312 may be a 3D display. The speaker (or speakers) 314 may be provided as integrated with other elements of electronic device 300 or may be a stand-alone unit. In some embodiments, the display 312 may be outputted through speaker 314.

The equipment device 300 of FIG. 3 can be implemented in system 200 of FIG. 2 as primary equipment device 202, but any other type of user equipment suitable for allowing communications between two separate user devices for performing the functions related to detecting sensors and electronic devices with sensors within proximity of a display device, determining the type of sensor, such as whether it is embedded in a smart glasses, smart watch, EKG monitor, blood sugar monitor etc., obtaining sleep stage associated with a user wearing the electronic device with the sensor, when multiple users are present, then obtaining sleep stage associated with multiple users wearing the electronic device with the sensor, applying filters to media asset or a portion/segment of the media asset based on a determination of sleep state of a user(s) after it has been decoded, fetching preconfigured content for a particular sleep state when available, selecting one or more filters, performing alarm and device configurations, accessing user profiles, detecting and storing user behavior patterns as it relates to previous filters applied, changing ringtones, determining conference call setting and filters to be applied during a conference call if a second user adjacent to the first user is asleep, automatically applying device configurations, detecting changes in a sleep state, implementing machine learning (ML) and artificial intelligence (AI) algorithms, and all the functionalities discussed associated with the figures mentioned in this application.

FIG. 4 is a block diagram of an example of a system having multiple electronic devices with sensors, filter modules, and content item server(s) for detecting a sleep state and applying filters or fetching a content item that is suited for the detected sleep state, in accordance with some embodiments of the disclosure.

FIG. 4 is a block diagram of an example of a system having multiple electronic devices with sensors, filter modules, and content item server(s) for detecting a sleep state and applying filters or fetching a content item that is suited for the detected sleep state, in accordance with some embodiments of the disclosure. In this embodiment, the system includes a plurality of users, such as user 1 at 420, user 2 at 430, and user n at 440. An electronic device having a sensor is worn by each of the users 420-440. The system also includes a viewing device of display device 450 that is connected to a processing device 460 and a content server 410, which may be the same server 202 as in FIG. 2. All the components of the system may be connected to each other via network 400. The network 400 may comprise one or more network systems, such as, without limitation, an internet, the cloud, LAN, Wi-Fi or other network systems suitable for processing applications, accessing and obtaining sensor data from the electronic devices worn by users 420-440, send instructions to processing device 460, obtain content from content server and stream it to the processing device 460, and implement filters at the content server.

In some embodiments, a media stream is used to transport a media asset from a content server 410 via network 400 to the processing device 460. At the processing device 460, the media stream is received by a decoder of the processing device. The media asset may be decoded segment by segment by the decoder. A segment that is decoded may be sent to the display device 450 for display. Once a sleep state is detected, a filter may be applied to the next segment at a processing device 460 and then sent to the display device for displaying the segment with the filter applied. The type of filter to be applied may be determined based on sleep state data received from the electronic device worn by the user 420-440. When multiple users are detected within a predetermined distance of each other, then sleep state of the multiple users may be considered to determine a filter.

In some embodiments, if a determination is made that the processing device 460 does not have local filtering capability, i.e., the filter that is associated with a determined sleep state cannot be applied by the processing device due to its lack of capabilities, then filter may be applied at the content server 410 and then transmitted to the display 450 via the network 400 and the processing device 460.

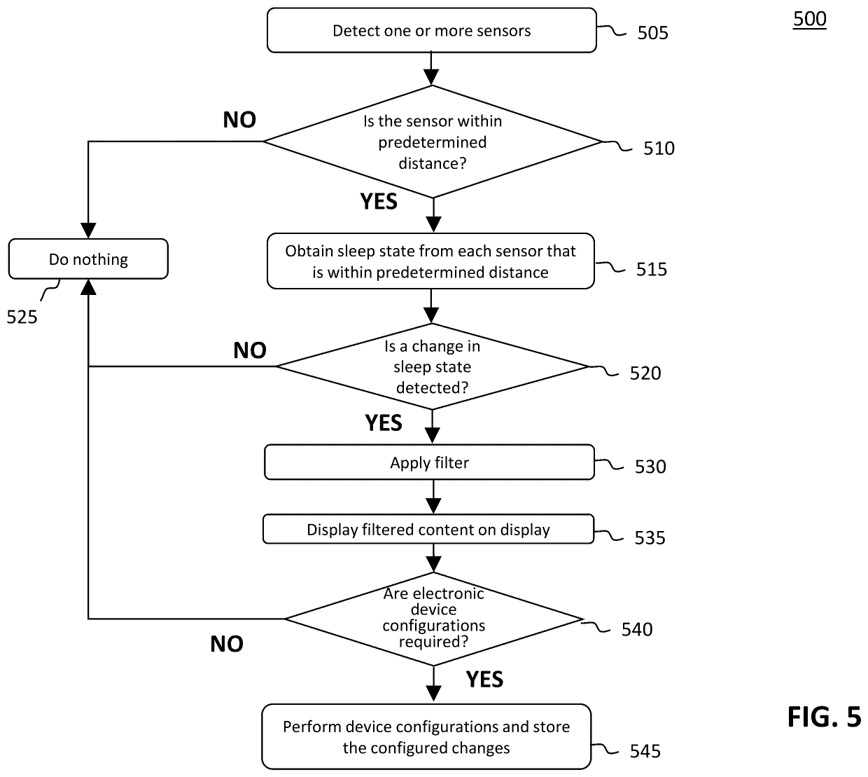

FIG. 5 is a flowchart of a process for detecting a sleep state and applying filters or fetching a content item that is suited for the detected sleep state, in accordance with some embodiments of the disclosure.

The process 500 may be implemented, in whole or in part, by systems or devices such as those shown in FIGS. 2-4. One or more actions of the process 500 may be incorporated into or combined with one or more actions of any other process or embodiments described herein. The process 500 may be saved to a memory or storage (e.g., any one of those depicted in FIGS. 2-4) as one or more instructions or routines that may be executed by a corresponding device or system to implement the method 500.

In some embodiments, at block 505, the system, such as the system in FIG. 2, via its control circuitry 220 and/or 228, detects one or more sensors. These may be sensors that are associated with one or more electronic devices. For example, a sensor associated with a smart watch worn by a user may be detected. The sensor may also be a sensor housed in a smart glasses, Wi-Fi earbuds, a smart watch, a smart belt, a heart monitor, an EKG monitor, a blood sugar monitor, or any other type of sleep sensor in any other type of monitoring device or sleep monitoring device. The sensor may also be in any other type of electronic device or equipment that can measure brain waves, breathing, heart rate, body movement, eye movement, blood sugar oxygen levels, and any other movement or data that can be used to determine a sleep state of a user wearing the electronic device.

When multiple sensors are detected, that may be indicative of multiple users wearing multiple electronic devices that include sensors. In some embodiments, the detection of single sensor, multiple sensors, or a set of sensors within one electronic device may trigger, based on the processing device, establishing a connection with the electronic device, such as via the electronic device logging in to the processing device or the associated display device. In other embodiments, the processing device may continuously monitor for an electronic device. It may do so by monitoring Bluetooth signals or Wi-Fi connections. The processing device may also use a camera associated with the display device to determine if any user wearing an electronic device has entered a room or space where the display device is located. The monitoring of electronic devices with sensors may be triggered by the processing device at all times, at predetermined intervals, or whenever an electronic device establishes a Bluetooth or Wi-Fi connection. Other methods of detecting wireless devices that include sensors are also contemplated within the embodiments.

At block 510, the control circuitry 220 and/or 228 may determine whether the sensor is within a predetermined distance or proximity to the processing device. The predetermined distance may be determined by the system based on the area surrounding a display device. For example, if a display device is in a bedroom, hotel room, lounge area, or in another interior or exterior location, the predetermined distance may be determined based on size of the bedroom, hotel room, lounge area, etc. For example, in a 10′×12′ room, the predetermined distance may be the longest dimension of the room, e.g., 12 feet. If the television is located in an area where the space is much larger, such as an outdoor seating area like a backyard or a large hall, then a fixed predetermined distance may be automatically selected, or another distance that is within the field of view from the display device may be selected.

In some embodiments, predetermined distance may include a distance beyond a current room or confined space. For example, in a multiple-sensor scenario, a first electronic device having a sensor may be located in the same room, and a second electronic device having a second sensor may be located in an adjacent room. As will be further described in the multiple-electronic devices/sensors scenarios below, the predetermined distance may include, in some embodiments, distance that covers multiple adjacent rooms such that a TV in a first room used by a user of the first electronic device is not loud enough to disturb a second user who is wearing a second electronic device with a sensor, is in an adjacent room, and is currently asleep.

Whatever the predetermined distance is, at block 510, if a determination is made that the electronic device is outside the predetermined distance, then, at block 525, the processing device may not perform any steps to determine sleep state. An electronic device that is detected outside the predetermined distance may be indicative of a device that is far away from the display device and not associated with a user that is consuming a media asset on the display device associated with the processing device.

At block 510, if a determination is made that the electronic device is within the predetermined distance, then, at block 515, the processing device may obtain the sleep state from each sensor (i.e., each electronic device) that is within the predetermined distance.

To obtain the sleep state, the processing device, via the control circuitry 220 and/or 228, may access the sensor in the electronic device or equipment worn by the user and obtain data relating to measurement of brain waves, breathing, heart rate, body movement, eye movement, blood sugar oxygen levels, and any other movement or data that can be used to determine a sleep state.

Based on the data received, the data may be correlated with a sleep state. For example, if the electronic device is a heart rate monitor, then the control circuitry 220 and/or 228 may access the heart rate monitor to obtain heart rate from it and heart or pulse rate may be correlated with the user's sleep state. Since heart rate is slower when a user is sleeping as compared to when the user is awake or performing an activity, even if the activity is simply sitting in bed, such heart rate data obtained may be used to determine whether the user is awake, asleep or in various degrees of awareness and sleepiness as depicted in one example of a sleep state scale in FIG. 7. If the control circuitry 220 and/or 228 applies an actigraphy process, which is a method to detect movement or heart rate, such as by using a smart watch to detect the user's movement, data from such smart watch may be correlated with a sleep state. The data may also be from a pair of smart glasses, such as from its inward-facing camera. Such data may include the user's gaze, dilation in the user's eyes, eye pigmentation, degree of attention, frequency of eyes closing, or water content in the eyes, and such data may be correlated with their sleep state. Other methods of obtaining data and correlating it with a sleep state are described above in connection with block 101 of FIG. 1.

At block 520, the control circuitry 220 and/or 228 may determine whether there is a change in a sleep state. Determining change allows the control circuitry 220 and/or 228 to change a previously applied filter at the processing device to accommodate for the most current sleep state. For example, if a certain filter is applied when the user is feeling tired and sleepy, and then subsequently a change in sleep state in which the user has fallen asleep is detected, then another filter that is associated with the user's asleep sleep state may be applied. As such, the control circuitry 220 and/or 228 may monitor sleep states and changes in sleep states such that filters may be changed to accommodate new sleep states.

In one embodiment, the control circuitry 220 and/or 228 may track the increase or decrease of heart rate over time and correlate that with a change in sleep state. The heart rate slowing to its slowest from a moderate heart rate, for example, may be indicative of the user transitioning from sleep to deep sleep or REM sleep state, which has the lowest heart rate.

The sleep state may be quantified in a variety of scales. In some embodiments, the sleep scale may range from 1-10, 1-100, A-Z or some other numbering system. In other embodiments, as depicted in FIG. 7, the sleep scale may be a range from wide awake to deep sleep. There may be several degrees and ranges of sleep states within the scales described herein. There may also be several substages, which are transition stages between each sleep state.

Once a change in sleep state is detected, which may be from any one level in the sleep scale to the next level, such as from awake to feeling tired, at block 530, the control circuitry 220 and/or 228 may apply a filter that correlates with the changed sleep state.

The application of the filter, also referred to herein as application after decoding of media asset or portion of media asset or post processing, is performed by the processing device associated with the display device. Although reference is made here to application of filter after decoding of media asset or portion of media asset, the application of the filter may be in real-time or in the midst of a segment being decoded.

Some examples of filters applied are depicted in FIGS. 8-12 and 17-18. This would mean that the media asset is filtered at the processing device before it is displayed on the display device, such as a TV, that is connected to the processing device. For example, if a transition from an awake state to a sleep state is determined, then a filter that lowers the volume and brightness may be applied at the processing device such that, once it is applied, the resulting display of the media asset on the display device is displayed with a lower volume and brightness.

In some embodiments, the process of applying a filter may include receiving a media stream associated with a media item by a decoder of the processing device. The media asset may be decoded segment by segment by the decoder. The segment that is decoded may be sent to the display device for display. In one embodiment, once a sleep state or change in sleep state is detected, a filter may be applied to the next segment, such as by the processing device or a content item server, once it is decoded but not yet sent to the display device. The type of filter to be applied may be determined based on sleep state data received from the electronic device worn by the user.

In another embodiment, the media asset may be decoded and immediately after decoding, and before rendering, a filer may be applied. The application of the filter may be performed in real-time in some embodiments. The application of the filter may also be performed in the midst of a segment being decoded such that the system may not need to wait for the completion of the entire segment to be decoded. The type of filter to be applied may be determined based on sleep state data received from the electronic device worn by the user or another type of electronic device that is not worn by the user and can perform monitoring or sensing of the user.

An example of a filter applied may include changes to hue, blue light, brightness, sound, selection of sounds, closed captioning, ringtone, sunset/sunrise, haptic mode, white noise, and alarm. For example, if a filter that eliminates a certain sound in the media asset, activates closed captioning, or eliminates blue light in the media asset is determined to be the best filter that correlates with the changed sleep state, then such filter(s) will be applied.

The filters are applied by the processing device and not the display device. By doing so, even if the television does not have a capability to perform certain operations, e.g., eliminate blue light, etc., which may be due to the television being an older model, the control circuitry 220 and/or 228 may still accomplish the end result by applying the associated filter at the processing device to cause the media asset to be displayed in the desired manner, e.g., with the blue light eliminated.

At block 535, once the filter is applied at the processing device, the media segment with the filter applied may be displayed on the display device. For example, if a user is watching a movie and has consumed the first 33 minutes, once a sleep state is determined, the portion of the movie that sequentially follows the 33-minute mark may be displayed with the filter applied. The control circuitry 220 and/or 228 may continue to track changes in sleep state and continue to apply additional filters or adjust the degree of the applied filter to match the changing sleep state.

At block 540, a determination may be made if electronic device configurations are required. In other words, are configurations to the electronic device worn by the user required? If so, at block 545, the control circuitry 220 and/or 228 may perform device configurations as needed. In some embodiments, the device configurations that can be applied include changing the time of an upcoming alarm, changing future settings of the device such as all subsequent alarms, changing the ringtone, updating the user profile based on sleep states determined, and detecting and storing sleep patterns. Although some examples of device configurations filters are depicted at block 545, the embodiments are not so limited. Additional types of device configurations are also contemplated in the embodiments. If device configuration is not required, then from block 540, the process may return to block 525, where no further actions that relate to electronic device configurations are taken.

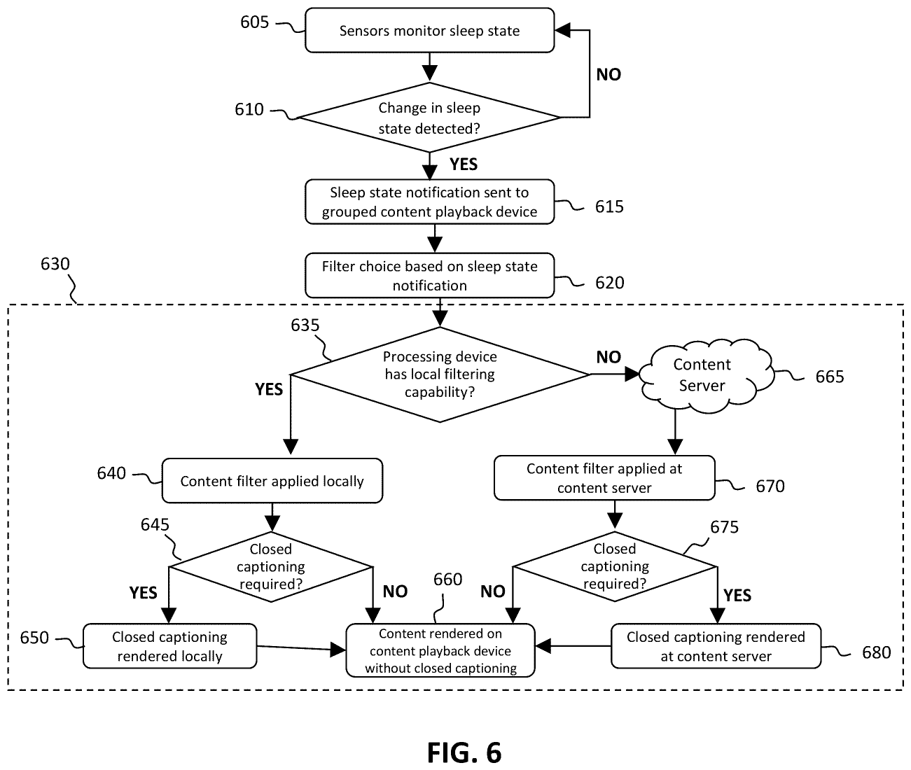

FIG. 6 is a flowchart of a process for detecting a sleep state and applying filters either locally or at server level based on the detected sleep state, in accordance with some embodiments of the disclosure.

The process 600 may be implemented, in whole or in part, by systems or devices such as those shown in FIGS. 2-4. One or more actions of the process 600 may be incorporated into or combined with one or more actions of any other process or embodiments described herein. The process 600 may be saved to a memory or storage (e.g., any one of those depicted in FIGS. 2-4) as one or more instructions or routines that may be executed by a corresponding device or system to implement the method 600.

In some embodiments, at block 605, the system, such as the system in FIG. 2, via its control circuitry 220 and/or 228, monitors a sleep state. The monitoring performed is the monitoring of a sensor in an electronic device worn by the user. For example, the monitoring can be of a sensor associated with a smart watch worn by the user. It may also be of a sensor housed in a smart glasses, Wi-Fi earbuds, a smart watch, a smart belt, a heart monitor, an EKG monitor, a blood sugar monitor, or a sleep sensor in a sleep monitoring device.

The monitoring may include monitoring, obtaining data related to, or measuring, brain waves, breathing, heart rate, body movement, eye movement, blood sugar oxygen levels, and any other movement or other data associated with a user of the electronic device that can be used to determine a sleep state of the user. In embodiments in which multiple electronic devices are detected by the system, all the electronic devices that are within a predetermined distance may be monitored.

In some embodiments, the monitoring may be triggered when the processing device establishes a connection with the electronic device. In other embodiments, the monitoring may be constant and ongoing to determine if any electronic devices are present within the predetermined distance of the processing device and to obtain a sleep state from such electronic devices.

At block 610, a determination is made whether a change in sleep state is detected. To detect a change in sleep state, the control circuitry 220 and/or 228 may track a sleep state, based on data received from a sensor of an electronic device worn by a user, and compare sleep state values on a periodic basis to determine if a change in sleep state has occurred. For example, if a sleep scale that ranges from 1-10, 1-100, or A-Z is used, then a change in sleep state may be determined if the subsequent reading from a sensor from the electronic device correlates with a sleep state of 7, 38, or C in the 1-10, 1-100, or A-Z scale and the previous reading was a 6, 37, or a B. In other words, an increase from 6 to 7, 37 to 38, and B to C, respectively, in the scale used may be associated with a change in sleep state. These values in sleep scale may also correlate to states of sleep, such as awake, tired, sleepy, light sleep, deep sleep, waking up, fully awake, etc. Although an example of a sleep scale is described, the embodiments are not so limited and other sleep scales, including the sleep scale depicted in FIG. 7, may also be used.

In some embodiments, the control circuitry 220 and/or 228, to determine whether a change in sleep state has occurred, may track increase or decrease of heart rate over time and correlate that with a change in sleep state. The heart rate slowing to its slowest from a moderate heart rate, for example, may be indicative of the user transitioning from sleep to deep sleep state or REM sleep state, which has the lowest heart rate.

At block 610, if a change in sleep state is not detected, then the process may revert back to block 605, where the control circuitry may continue to monitor the sensor in the electronic device until a sleep state change is detected.

At block 610, if a change in sleep state is detected, then the control circuitry 220 and/or 228, at block 615, may send a sleep state notification to a grouped content item playback device, such as to a common television that is used by the group or to the processing device associated with the television. The grouped content item playback device is a device that is shared by a group, not just an single individual, such as the group being a family and the device being a television that is in a living room and shared by all members of the family.

At block 620, based on the type of sleep state, or change in sleep state, detected, the control circuitry 220 and/or 228 may determine a filter choice. Some examples of filters choices are depicted in FIGS. 8-12 and 17-18.

The process of selecting and applying the filter is described in section 630, which includes blocks 635-680. At block 635, the control circuitry 220 and/or 228 may determine whether the processing device has local filtering capability. The processing device may be any devices such as a set-top box, casting stick, USB stick, streaming stick, or another device capable of applying a filter post decode. Some examples of processing devices that can be used in some embodiments to add the unique filtering embodiments discussed herein based on sleep state include TiVo Edge™, Chromecast™ with Google TV™, Roku™ streaming stick, FireTV™ stick, Anycast™ streaming stick, Apple TV™, Nvidia Shield™, and Amazon Fire TV Cube™. The processing device, which may be connected to the content item playback device may perform the process of adding the filters. As referred o herein, the display device may be, in one embodiment, television. In other embodiments, it may be any display device.

If a determination is made, at block 635, that the processing device has local filtering capability, then the process may proceed to blocks 640-660. However, if a determination is made, at block 635, that the processing device does not have local filtering capability, i.e., the filter that is associated with a determined sleep state cannot be applied by the processing device due to its lack of capabilities, then the process may proceed to blocks 665-680.

If the processing device has local filtering capability, then, at block 640, the content item filter is applied at the processing device. If the filter to be applied is closed captioning and a determination is made at block 645 that the filter is required, then the processing device may apply the closed captioning filter at block 650 and render the media asset with the closed captioning filter. However, if a determination is made at block 645 that the closed captioning filter is not required, then the processing device may render the media asset on the playback device, such as a television, at block 660, without the closed captioning filter.

If a determination is made, at block 635, that the processing device does not have local filtering capability, then a content item server may be used at block 665. The content item server, instead of the processing device, may apply the filter, at 670, that is associated with a determined sleep state.

In some embodiments segments of the media asset may be encoded which have different levels of sleep state along with their bit rates. For example, a first segment may be associated with a first sleep state, such as drowsy, sleepy, or tired, and a second segment may be associated with another sleep state that is distinct than the first sleep state, such as asleep or deep in REM sleep. Such different sleep states may be represented in a manifest file. The processing device, or the content server when the processing device is not capable of applying the filter, may request the segment based on available bandwidth and the proper sleep state. In some scenarios, the requested segment may be re-encoded at the same bit rate.

At block 675, a determination is made whether closed captioning is required based on the determined sleep state. If a determination is made that the closed captioning filter is required, then the content item server may apply the closed captioning filter, at block 680, and render the media asset with the closed captioning filter. However, if a determination is made, at block 675, that the closed captioning filter is not required, then the content item server may render the media asset on the playback device, such as a television, at block 660, without the closed captioning filter.

FIG. 7 is a block diagram of an example of a sleep state scale used by the system, in accordance with some embodiments of the disclosure. As described above, a variety of sleep scales and denomination in the sleep scale may be used. In one embodiment, the sleep scale used may range from wide awake on one side of the spectrum to deep sleep on the other, as depicted at 710 of FIG. 7. In some embodiments, the sleep scale may range from 1-10, 1-100, A-Z or some other numbering system.

FIG. 8 depicts an example of a scenario in which the sensor detects a long period of an awake state, in accordance with some embodiments of the disclosure. In this embodiment, the control circuitry 220 and/or 228 receives data from a sensor of an electronic device worn by the user. The data may include biometric and other sensor data. The electronic device may be smart glasses, Wi-Fi earbuds, a smart watch, a smart belt, a heart monitor, an EKG monitor, a blood sugar monitor, a sleep sensor in a sleep monitoring device, or any other electronic device that is capable of performing biometric readings that can be used to determine a sleep state.

The data received may be processed by the control circuitry 220 and/or 228 and associated with a sleep state. For example, data received may be data related to heart rate. The control circuitry 220 and/or 228 may associate the received heart rate with a sleep state, such as awake, tired, asleep or other states. If a low heart rate data is received, such as heart rate between 40-50 beats per minute, then such heart rate may be associated with an asleep state.

In FIG. 8, the data received by the control circuitry, in one embodiment, relates to long periods of an awake state. For example, the data may indicate constant motion, higher heart beats, wide awake eyes, and other such data from the sensors that can be associated with the user being awake. Based on the awake state determined, the action taken, or the filter applied, may be to display the media asset at its high brightness setting, at high sound, and without closed captioning. As such, the control circuitry 220 and/or 228 may transition from the media asset being displayed on the television 800 to applying a filter that has a higher brightness, higher sound, and no closed captioning since the user is in an awake state.

In another embodiment, the control circuitry 220 and/or 228 may access a profile of the user consuming the media asset to determine if there is a type of filter the user prefers, or the setting the user prefers, when in the awake state. The profile may have been created by the user or by the control circuitry 220 and/or 228 based on historical data from execution of machine learning algorithms that detect pattern in which a similar scenario of sleep state occurred and then either the user applied a certain setting to their display device or the control circuitry 220 and/or 228 applied a filter at the processing device. The control circuitry 220 and/or 228 may access the profile and if such a preference for an awake state exists, use the preference in determining which filter to apply at the processing device.

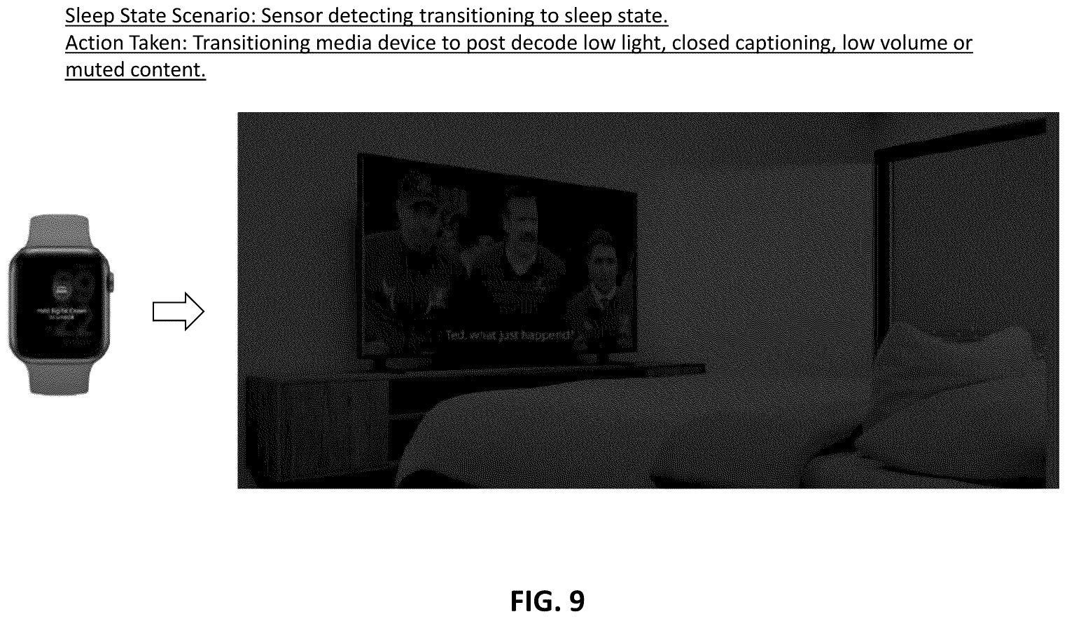

FIG. 9 depicts an example of a scenario in which the sensor detects transitioning to a sleep state, in accordance with some embodiments of the disclosure. In this embodiment, the control circuitry 220 and/or 228 receives data from a sensor of an electronic device worn by the user. The electronic device may be any device that includes a sensor that is capable of taking biometric readings that can be used to determine a sleep state.

The data received may be processed by the control circuitry 220 and/or 228 and associated with a sleep state. The data received by the control circuitry, in one embodiment, as depicted in FIG. 9, relates to a transition to a sleep state. When the change in a sleep state is detected, in one embodiment, the control circuitry 220 and/or 228 at the processing device may apply a filter that is designated for the transition to sleep state.

Based on the transition to a sleep state, the action taken, or the filter applied, may be to lower the light, invoke closed captioning, and lower or mute the volume. As such, the control circuitry 220 and/or 228 may apply the filters associated with lower the light, invoke closed captioning, and lower or mute the volume at the processing device and display the media asset on the display device after the filters have been applied.

As mentioned earlier, the control circuitry 220 and/or 228, in some embodiments, may access a profile of the user consuming the media asset to determine if the user has any preferences on the type of filter the user prefers, or the setting the user prefers, when the user is transitioning to a sleep state. The profile may have been created by the user or by the control circuitry 220 and/or 228 based on historical data from execution of machine learning algorithms that detect a pattern in which a similar scenario of a sleep state occurred and either the user applied a certain setting to their display device or the control circuitry 220 and/or 228 applied a filter at the processing device. The control circuitry 220 and/or 228 may access the profile, and if such a preference for transitioning to sleep state exists, use the preference in determining which filter to apply at the processing device.

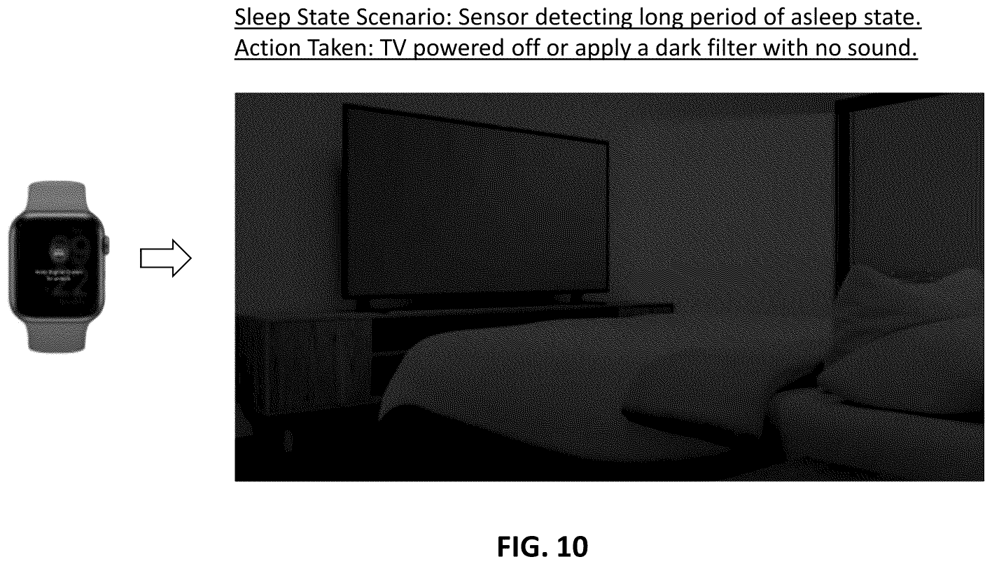

FIG. 10 depicts an example of a scenario in which the sensor detects a long period of an asleep state, in accordance with some embodiments of the disclosure. In this embodiment, the control circuitry 220 and/or 228 receives data from a sensor of an electronic device worn by the user. The electronic device may be any device that includes a sensor that is capable of taking biometric readings that can be used to determine a sleep state.

The data received may be processed by the control circuitry 220 and/or 228 and associated with a sleep state. The data received by the control circuitry, in one embodiment, as depicted in FIG. 10, relates to long periods of an asleep state. What is considered a long period may be defined by the control circuitry 220 and/or 228, the user, or based on crowdsourced data from other users. For example, a predetermined number, such as two hours of sleep with movement below a threshold or heart rate below a threshold may be used to determine whether the user's sleep state meets the criteria for being asleep for a long period of time. The data may vary for what is considered a long period of being asleep and be based on the user and case by case, for example the user's sleep patterns, their age, demographic, medical condition, amount of work perform prior to going to sleep (such as long day of work, late night of work, going to bed late, just ran a marathon, etc.) may be considered in determining what is a long period of being awake for that specific user.

When the change in sleep state is detected, the control circuitry 220 and/or 228, in one embodiment, at the processing device, may apply a filter that is designated for the long state of being asleep. The filter, in some embodiments, for this state of sleep may include a filter that darkens the display such that no visible light is displayed (or minimum visible light) and the sound is turned off. In other words, the control circuitry powers off the display device but by applying the filter rather than actually using the display device's controls. In other embodiments, the action taken may be to power off the display device using the display device's controls based on the long state of being asleep.

As mentioned earlier, the control circuitry 220 and/or 228, in some embodiments, may access a profile of the user consuming the media asset to determine if the user has any type of filter the user prefers, or the setting the user prefers, when the user is in a long state of being asleep. The profile may have been created by the user or by the control circuitry 220 and/or 228 based on historical data from execution of machine learning algorithms that detect a pattern in which a similar scenario of sleep state occurred and either the user applied a certain setting to their display device or the control circuitry 220 and/or 228 applied a filter at the processing device. The control circuitry 220 and/or 228 may access the profile and, if such a preference for long period of asleep state exists, use the preference in determining which filter to apply at the processing device. For example, the user may prefer, as indicated by the user's profile, that white noise be turned on when a long period of asleep state is detected. As such, a filter that includes white noise may be applied by the control circuitry 220 and/or 228 at the processing device.

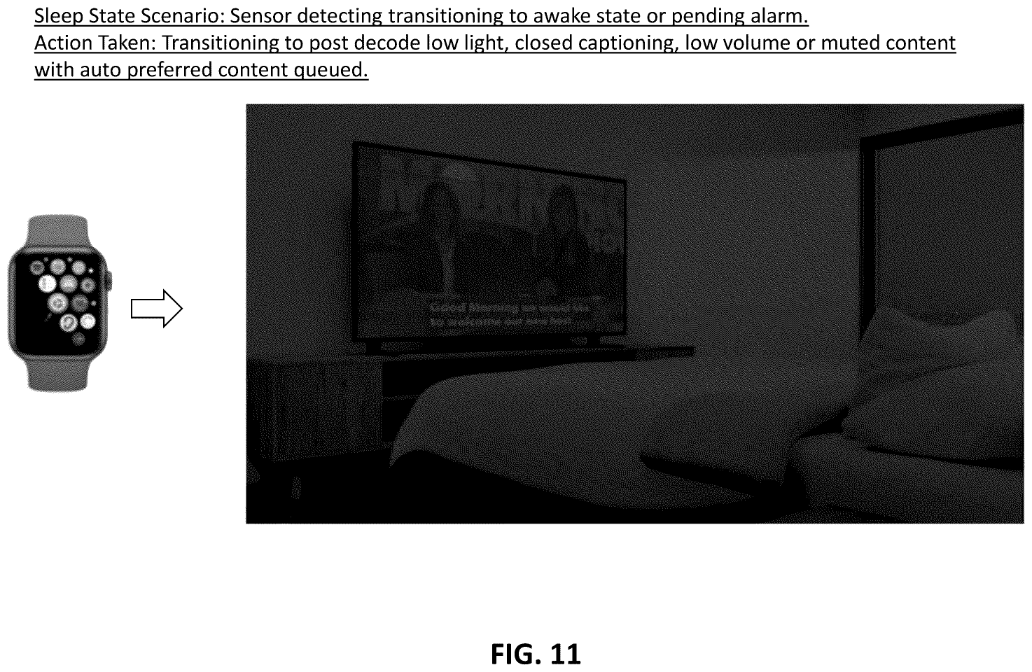

FIG. 11 depicts an example of a scenario in which the sensor detects transitioning to awake state or pending alarm, in accordance with some embodiments of the disclosure.

In this embodiment, the control circuitry 220 and/or 228 receives data from a sensor of an electronic device worn by the user. The data received may be processed by the control circuitry 220 and/or 228 and associated with a sleep state. The data received by the control circuitry, in one embodiment, as depicted in FIG. 11, relates to transitioning to an awake state. The data may also indicate that an upcoming alarm will occur in a few seconds, minutes, etc.

In the scenario, the state of transitioning to an awake state is determined by, for example, the user starting to make movement, heart rate increasing, user moving into a posture that is typical of the user prior to being awake. Data from sensors that are on the user's body, or other sensors and cameras, may be used to determine such a sleep state. For example, a smart watch, and other worn electronics as described in block 101 of FIG. 1, may be used to detect the user's movements and heart rate while cameras in the room or sensors that are in a bed may be used to determine user posture.

When the change in sleep state is detected, the control circuitry 220 and/or 228, in one embodiment, at the processing device, may apply a filter that is designated for the transitioning to awake state. The filter, in some embodiments, for this state of sleep may include low light, closed captioning, low volume or muted volume, with auto preferred content item queued to be displayed. As referred to herein, auto preferred content item may be a content item that the user desired to consume on a regular basis and has listed the item in the user's profile as preferred item to consume based on the sleep state.

The filter may allow the user to transition into the awake state with a display with lesser light luminosity (e.g., below a predetermined threshold) and volume transition so as not to shock or alarm the user with bright lights or loud sounds when the user is still getting out of sleep. As such, the embodiments may also include filtering out any loud sounds in the media asset displayed. For example, if the media asset includes sounds of a fire engine siren, as background or ambient sound, in a scene of the media asset, the filter may tune out that particular siren sound and minimize any other loud sound.

As mentioned earlier, the control circuitry 220 and/or 228, in some embodiments, may access a profile of the user consuming the media asset to determine if the user has any preferences on the type of filter the user prefers, or the setting the user prefers, when in the transitioning to awake state. The profile may have been created by the user or by the control circuitry 220 and/or 228 based on historical data from execution of machine learning algorithms that detect a pattern in which a similar scenario of sleep state occurred and either the user applied a certain setting to their display device or the control circuitry 220 and/or 228 applied a filter at the processing device. The control circuitry 220 and/or 228 may access the profile and, if such a preference for transitioning to awake state exists, use the preference in determining which filter to apply at the processing device.

The control circuitry 220 and/or 228 may also determine that a transition to an awake state will occur soon based on an alarm setting of the user. For example, the user may have set a 6:30 AM alarm for wake up. The alarm may be set on the user's smart watch or any of the electronic devices described in block 101 of FIG. 1. The alarm may also be set on a separate device that is not worn by the user, such a smart clock at the bedside of the user or any other smart electronic device in the room where the user is sleeping. The control circuitry 220 and/or 228 may access all such devices to determine if an alarm is set and if so, determine the wake-up time set by the alarm. Determining that an alarm is set, by accessing the smart device, either worn by the user or in the same room, the control circuitry 220 and/or 228 may trigger the transitioning to awake state routine, such as low light, closed captioning, low volume or muted volume, with auto preferred content item queued to be displayed, and tuning out any loud noises in the media asset.

In some embodiments, if the user hits a snooze button while waking up, the control circuitry 220 and/or 228 may modify the wake-up routine to allow more time for the user to transition to the wake state. In other embodiments, if the control circuitry 220 and/or 228 determines, such as based on a user profile, that the wake-up time is essential and cannot be delayed despite the snooze alarm, then the control circuitry 220 and/or 228 may trigger the wake-up routine despite the alarm. For example, such a determination may be made based on the control circuitry 220 and/or 228 determining that the user has to make it to a meeting or has to catch a train that leaves at a particular time. Such data may be accessed by the control circuitry 220 and/or 228 from the user's profile or based on any emails, texts, social media posts, calendars and other documents exchanged by the user that are accessed by the control circuitry 220 and/or 228.

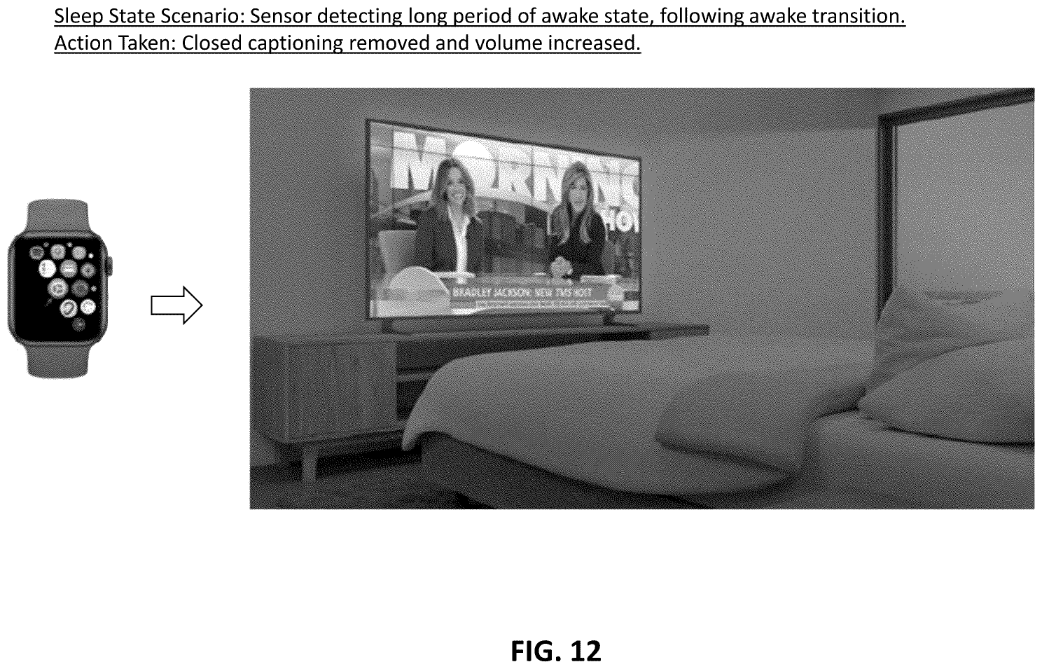

FIG. 12 depicts an example of a scenario in which the sensor detects a long period of awake state, following awake transition, in accordance with some embodiments of the disclosure. In this embodiment, the control circuitry 220 and/or 228 receives data from a sensor of an electronic device worn by the user. The data received may be processed by the control circuitry 220 and/or 228 and associated with a sleep state, such as in FIG. 12, and a long period of awake state that followed a transition to awake state. In other words, the user had gotten up from sleep a while back and then has been awake for a long period. What is considered a long period of awake state may be defined by the control circuitry 220 and/or 228, the user, or based on crowdsourced data from other users. For example, a predetermined number, such as three hours of being awake may be predetermined to be a long period of being awake. The data may vary based on the user and on a case-by-case basis. For example, the user's sleep patterns, their age, demographic, medical condition, amount of work performed prior to going to sleep (such as long day of work, late night of work, going to bed late, just ran a marathon, etc.) may be considered in determining what is a long period of being awake for that specific user.

When the change in sleep state is detected, the control circuitry 220 and/or 228, in one embodiment, at the processing device, may apply a filter that is designated for the long state of being awake. The filter, in some embodiments, for this state of sleep may be a filter that has closed captioning removed and the brightness and volume are at their highest, or whatever volume and brightness are preferred by the user in this sleep state, as indicated in their profile.

The embodiments are not limited to different sleep states described in FIGS. 8-12. Any other sleep state is also contemplated in the embodiments. Likewise, the filters applied to different sleep states are also not limited, and any other type of filter may be applied by the control circuitry 220 and/or 228 based on a pattern of prior used filters, user profile, or crowdsourced data on what profiles are preferred by other users with same age, demographic, location, medical condition, gender, etc. The filters may also be customized for each user based on an activity previously performed by the user, such as the user had a long day of work, had late night of work, was traveling on a long flight, went to bed late, was binge watching movies the previous night, ran a marathon the day before, recovered from an illness or is still undergoing an illness, went to a bar the night before, and several other factors. All such data may be obtained by the control circuitry 220 and/or 228 from the user's profile or based on any emails, texts, social media posts, calendars and other documents exchanged by the user that is accessed by the control circuitry 220 and/or 228 and processed by an artificial intelligence engine running an artificial intelligence algorithm.

The examples of filters applied in FIGS. 8-12 may be applied either at the processing device or at the content item server. The filters are applied to the media asset, or the next portion or segment of the media asset, prior to the media asset or its segment being displayed on the display device, such as the smart television. The filters applied to the media asset are also applied without changing or configuring the controls of the display device. In other words, the filters are applied at the processing device or at the content item server but without the user manipulating the display device controls directly or indirectly. Even if the display device does not have the capability of changing a certain setting, such as removing selected background sounds and ambient noise, removal of such selected background sounds and ambient noise is handled by the system at the processing device or content item server by applying the appropriate filter.

FIG. 13 is a flowchart of a process for detecting a sleep state when multiple sensors are present and applying filters based on the rules associated with multiple sensors, in accordance with some embodiments of the disclosure.

The process 1300 may be implemented, in whole or in part, by systems or devices such as those shown in FIGS. 2-4. One or more actions of the process 1300 may be incorporated into or combined with one or more actions of any other process or embodiments described herein. The process 1300 may be saved to a memory or storage (e.g., any one of those depicted in FIGS. 2-4) as one or more instructions or routines that may be executed by a corresponding device or system to implement the process 1300.

In this embodiment, multiple users, such as husband, wife, siblings, friends and others may be present in a same room or within a predetermined distance of a display device. The predetermined distance may be determined by the system based on the area surrounding the display device or defined by a user of the system. For example, if a display device is in a bedroom, hotel room, lounge area, or another interior or exterior location, the predetermined distance may be determined based on size of the bedroom, hotel room, lounge area, etc. For example, in a 10′×12′ room, the predetermined distance may be the longest dimension of the room, i.e., 12 feet. If the television is located in an area where the space is much larger, i.e., the multiple users are within a confined area of each other and in a larger space surrounding them, such as an outdoor seating area like a backyard or a large hall, then a fixed predetermined distance may be automatically selected, or another distance that is within the field of view from the display device may be selected.

In some embodiments, predetermined distance may include a distance beyond a current room or confined space. For example, if multiple users, with each user wearing an electronic device with a sensor, are located in rooms next to each other that are separated by walls, then the predetermined distance, in some embodiments, may include area that covers multiple adjacent rooms such that if a TV in a first room used by a first user is turned on, it should not disturb a second user in an adjacent room who is sleeping.

As depicted in blocks 1310 and 1340, in a multiple-user (i.e., multiple-sensor) scenario, the control circuitry 220 and/or 228 determines a sleep state of a first user and a second user. Although only two users are used to describe the embodiments of FIG. 13, the embodiments are not so limited and are applicable to any number of users.

The determination of the sleep state at 1310 and 1340 may be via monitoring a sensor in an electronic device worn by the user, such as monitoring of a first device associated with a first user having the first sensor and monitoring of a second device associated with a second user having the second sensor. For example, the monitoring can be of a sensor associated with a smart watch worn by the user. It may also be of a sensor housed in a smart glasses, Wi-Fi earbuds, a smart watch, a smart belt, a heart monitor, an EKG monitor, a blood sugar monitor, or a sleep sensor in a sleep monitoring device.

The monitoring may include monitoring, obtaining data related to, or measuring, brain waves, breathing, heart rate, body movement, eye movement, blood sugar oxygen levels, and any other movement or other data associated with a user of the electronic device that can be used to determine a sleep state of the user. In embodiments in which multiple electronic devices are detected by the system, all the electronic devices that are within a predetermined distance may be monitored.

In some embodiments, the monitoring may be triggered when the processing device establishes a connection with the electronic device of any of the multiple sensors. In other embodiments, the monitoring may be constant and ongoing to determine if any electronic devices are present within the predetermined distance of the processing device and to obtain a sleep state from such electronic devices.

At blocks 1320 and 1350, a determination is made whether a change in sleep state is detected either for the first sensor, which is associated with the first device associated with the first user, or the second sensor, which is associated with the second device associated with the second user. To detect a change in sleep state, the control circuitry 220 and/or 228 may track a sleep state, based on data received from a sensor of an electronic device worn by the first or second user, and compare sleep state values on a periodic basis with a previous value for the same user to determine if a change in sleep state has occurred. For example, if a sleep scale that ranges from 1-10, 1-100, or A-Z is used, then a change in sleep state may be determined if the subsequent reading from a sensor from the electronic device correlates with a sleep state of 7, 38, or C in the 1-10, 1-100, or A-Z scale and the previous reading was a 6, 37, or a B. In other words, an increase from 6 to 7, 37 to 38, and B to C, respectively, in the scale used may be associated with a change in sleep state. These values in sleep scale may also correlate to states of sleep, such as awake, tired, sleepy, light sleep, deep sleep, waking up, fully awake, etc. Although an example of a sleep scale is described, the embodiments are not so limited, and other sleep scales, including the sleep scale depicted in FIG. 7, may also be used.

In some embodiments, the control circuitry 220 and/or 228, to determine whether a change in sleep state has occurred, may track increase or decrease of heart rate over time and correlate that with a change in sleep state. The heart rate slowing to its slowest from a moderate heart rate, for example, may be indicative of the user transitioning from sleep to deep sleep state or REM sleep state, which has the lowest heart rate.

At blocks 1320 and 1350, if a change in sleep state is not detected, then the process may revert back to blocks 1310 and 1340, respectively, where the control circuitry may continue to monitor the first and the second sensor in the first and second electronic devices until a sleep state change is detected. In some embodiments, a change in sleep state is not detected for the first sensor while a change in sleep state is determined for the second sensor or vice versa. The process would proceed to next block if the change in sleep state is detected for the sensor and revert back to previous block if it is not detected.

At blocks 1320 and 1350, if a change in sleep state is detected, then the control circuitry 220 and/or 228, at blocks 1330 and 1360, may send a sleep state notification to a grouped content item playback device, such as to a common television or processing device associated with the television.

At block 1360, based on the type of sleep state, or change in sleep state, detected, the control circuitry 220 and/or 228 may determine a filter choice. Some examples of filters choices are depicted in FIGS. 8-12 and 17-18.

In some instances, the processing device may include the capability to apply a filter. In such embodiments, the content item filter is applied at the processing device, as depicted at block 1370. The processing device may be any devices such as a set-top box, casting stick, USB stick, streaming stick, or another device capable of applying a filter post decode. Some examples of processing devices that can be used in some embodiments to add the unique filtering embodiments discussed herein based on sleep state include TiVo Edge™, Chromecast™ with Google TV™, Roku™ streaming stick, FireTV™ stick, Anycast™ streaming stick, Apple TV™, Nvidia Shield™, and Amazon Fire TV Cube™. The processing device, which may be connected to the content item playback device, also referred to herein as the display device, such as a television, may perform the process of adding the filters.

In other embodiments, the processing device may not have the filtering capability. In such embodiments, a content item server may be used, instead of the processing device, to apply the filter that is associated with a determined sleep state, as depicted at block 1380. The process of applying the filter 1380, whether at the processing device or the content item server, is further described in block 630 of FIG. 6.

In yet other embodiments segments of the media asset may be encoded which have different levels of sleep state along with their bit rates. For example, a first segment may be associated with a first sleep state, such as drowsy, sleepy, or tired, and a second segment may be associated with another sleep state that is distinct than the first sleep state, such as asleep or deep in REM sleep. Such different sleep states may be represented in a manifest file. The processing device, or the content server when the processing device is not capable of applying the filter, may request the segment based on available bandwidth and the proper sleep state. In some scenarios, the requested segment may be re-encoded at the same bit rate.

In some embodiments, in a multiple-sensor environment where multiple sensors associated with multiple electronic devices are present within a predetermined distance of each other, application of a filter may be subject to additional multi-sensor rules. These rules may consider the preferences of more than two users present within the predetermined space in selecting a filter. For example, when two individuals, such as a couple, siblings, etc., are in the same bed, one user may be wide awake while another user may be asleep. As such, the control circuitry 220 and/or 228 may determine preferences of both users for their separate awake and asleep sleep states and then determine the best filter to be applied.

In selecting a filter when conflicting sleep states are determined, the control circuitry may determine preferences of each user for their respective sleep state and then determine whether their preferences violate another user's preferences based on their sleep state. For example, in one scenario, a first user may have a sleep state of being awake. The first user may have set a preference in their profile to play a selected media item when in the awake sleep state and to play it on the common television with a preferred high brightness and high volume setting. If the user is alone, then a filter that accommodates the first user's preferences in the awake state may be applied. However, in this scenario, since a second user is within a predetermined distance of the first user, such as in the same bed, and currently asleep, selecting a filter that suits only the first user may violate a preference of the second user who is asleep. The second user may have a preference to turn off any media device when they are fast asleep. In this situation, the control circuitry may determine a filter that can accommodate both users. The control circuitry may make the filter setting determination on its own or invoke an artificial intelligence and/or machine learning algorithm and select a filter based on results obtained from executing the algorithms.

In some embodiments, detecting the conflicting, different, or opposite sleep states of the two users, as described in the scenario above, the control circuitry 220 and/or 228 may select a filter that is a) a midway approach, b) suitable for the first user, c) suitable for the second user, d) a filter that accommodates both the first and the second user, or e) is based on priority given between the first and second user.

The midway approach may be an approach that selects a setting that is an average of the preferences of the first and second users. For example, if the first user prefers the volume at setting 9 for an awake sleep state, and the second user prefers it at a setting of 3 for waking up sleep state, then an average volume of 6 may be used for a filter.

The approach in which a filter that takes into consideration only either the first or second user's preferences for their sleep state may be used.

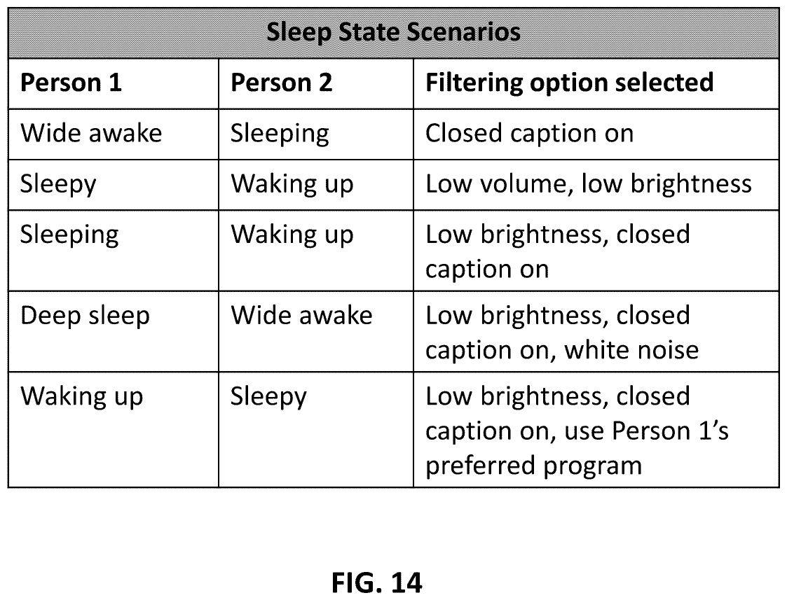

The approach in which a filter that accommodates both the first and the second user may also be used. In this approach, the control circuitry 220 and/or 228 may determine the best suitable filter taking into consideration both first and second user's sleep state. Some examples of this approach are provided in FIG. 14.

The approach based on priority may include the control circuitry 220 and/or 228 determining whether to prioritize the first of the second user's preferences in selecting a filter.

In some embodiments, the first user may be an adult and the second user may be a baby. Different priorities may be set for the adult and child combination, such as prioritizing the baby if the sleep state of the baby is asleep, so as not to wake up the baby.

In another embodiment, the first and second users may be husband and wife. The priority may be date dependent, such as priority may be given to the husband on certain days and the wife on certain days. For example, if the husband has meeting Monday and Tuesday and needs to be awake for the meeting and the wife has meetings on Wednesday and Thursday and needs to be awake for her meetings, then preferences from the husband's profile may be taken into consideration on Monday and Tuesday and preferences from the wife's profile may be taken into consideration on Wednesday and Thursday.

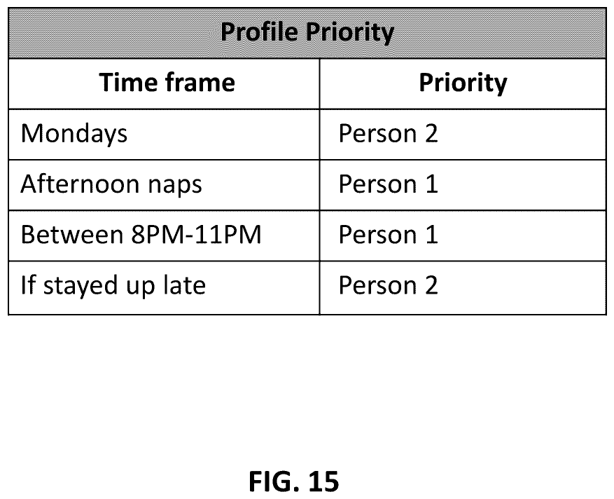

The priority, in some embodiments, may depend on date, time, day of week, whether an event is to occur for one of the users, tiredness level of the user, age, medical condition, holiday schedule, etc. For example, if the first user's current medical condition is that they are sick with a flu and need more rest, then their preferences may be prioritized over another user who is in the same room. Other examples of priority are provided in FIG. 15.

FIG. 16 is a block diagram of an example of inserting a preconfigured video clip into a media stream based on the sleep state detected, in accordance with some embodiments of the disclosure. Although a single sensor environment is described, the embodiments are not so limited and may apply in a multiple-sensor environment (e.g., multiple sensors associated with multiple user electronic devices within a predetermined distance from the display device).

The process 1600 relating to inserting a preconfigured video clip into a media stream based on the sleep state may be implemented, in whole or in part, by systems or devices such as those shown in FIGS. 2-4. One or more actions of the process 1600 may be incorporated into or combined with one or more actions of any other process or embodiments described herein. The process 1600 may be saved to a memory or storage (e.g., any one of those depicted in FIGS. 2-4) as one or more instructions or routines that may be executed by a corresponding device or system to implement the method 1600.

In some embodiments, a normal clip 1610 may be received by the control circuitry 220 and/or 228. The clip may be a video clip of a movie, drama, sitcom, documentary, or any other type of video clip. The received clip may be received via a media stream, and it may be a segment or a portion of the movie, drama, sitcom, documentary, or any other type of media asset.

Upon receiving the normal video clip, which may be a portion or segment of an entire media asset, the control circuitry 220 and/or 228 may use a decoder associated with the display device to decode the received normal clip. The media asset may be decoded segment by segment by the decoder. Each segment that is decoded may be sent to the display device for display.

The control circuitry 220 and/or 228 may detect a sleep state of a sensor, such as a single sensor or multiple sensors, during the playback of the normal video clip. For example, while consuming the normal clip that was decoded and sent to display for playback, a user who may have been tired may start falling asleep, and such change in the user's sleep state may be detected by a sensor housed in the device worn by the user, such as a user's smart watch. The sleep state change may be transmitted by the smart watch to the processing device or a content item server.

Once a sleep state or change in sleep state is detected, a filter may be applied to the next segment that follows the normal clip. The type of filter to be applied may be determined based on sleep state data received from the electronic device worn by the user. Some examples of filters applied are depicted in FIGS. 8-12 and 17-18.

Once a sleep state is determined, which may also be defined in a manifest file, the control circuitry 220 and/or 228 may signal to a content item server, such as the content item server 410 of FIG. 4, to provide the next portion/segment/clip that is associated with the determined sleep state. In this embodiment, a plurality of preconfigured portions/segments/clips for each sleep state may be premade and stored at the content item server or at a remote storage associated with the content item server. Once a sleep state is determined, a matching process may be performed to find a segment of the media asset that is premade for the determined sleep state. If such a version exists, then it may be injected into the media stream, received by the processing device, and then sent to the display device for display. The version may be downloaded by the processing device for a calculated bitrate and the determined sleep state. If such a version that matches the sleep state does not exist, then the current media stream may be played as is, with no changes, or a filter may be applied at the processing device as described above.

As depicted in FIG. 16, in some embodiments, the control circuitry 220 and/or 228 may detect that the current sleep state of a user, based on sleep state data received, is Sleep State A. Based on the sleep state, the control circuitry 220 and/or 228 may send the sleep state information to the content item server. The content item server may perform a matching process to determine if a premade clip for the determined sleep state exists. In some embodiments, a determination may be made that Clip A is the closest or best clip, or a clip that has been designated, for the determined sleep state. Accordingly, Clip A 1620 may be injected into the media stream and transmitted to the processing device for playing after the normal clip has ended. The server may use a clip determination module 1650 to find Clip A 1620. As such, Clip A that addresses the user's current sleep state may be played on the display device. In some embodiments, the user's preferences for Sleep State A may be obtained from the user's profile and taken into consideration by the content item server in selecting the clip to be injected into the media stream.

Once Clip A has been displayed, during its display, the control circuitry 220 and/or 228 may continue to monitor the sleep state and determine whether another change in sleep state has occurred. For example, from a tired sleep state, the user may have gone into a sleep state, and then again subsequently to a deep sleep state.

When Sleep State B is detected, which is a change in Sleep State A, the control circuitry 220 and/or 228 may send the sleep state information to the content item server. The content item server may perform a matching process to determine if a premade clip for the determined sleep state exists. In some embodiments, a determination may be made that Clip B is the closest or best clip, or a clip that has been designated, for the determined Sleep State B. Accordingly, Clip B 1630 may be injected into the media stream and transmitted to the processing device for playing after the Clip A has ended. The server may use a clip determination module 1650 to find Clip B 1630. As such, Clip B that addresses the user's current sleep state, such as being sleepy, may be played on the display device. In some embodiments, the user's preferences for Sleep State B may be obtained from the user's profile and taken into consideration by the content item server in selecting the clip to be injected into the media stream.

Once Clip B has been displayed, during its display, the control circuitry 220 and/or 228 may continue to monitor the sleep state and determine whether another change in sleep state has occurred. For example, from a sleepy sleep state, the user may have gone into a deep sleep state. When the deep sleep state is detected, the control circuitry 220 and/or 228 may send the sleep state information to the content item server and determine whether a premade clip for deep sleep exists. If it does, then the control circuitry 220 and/or 228 may fetch the clip allocated for deep sleep state, inject it into the media stream, and display it on the display device. For example, the filter applied in the clip for the deep sleep state may be to shut down the television, or lower the brightness and volume, turn on white noise, or any other type of filter and combination thereof.

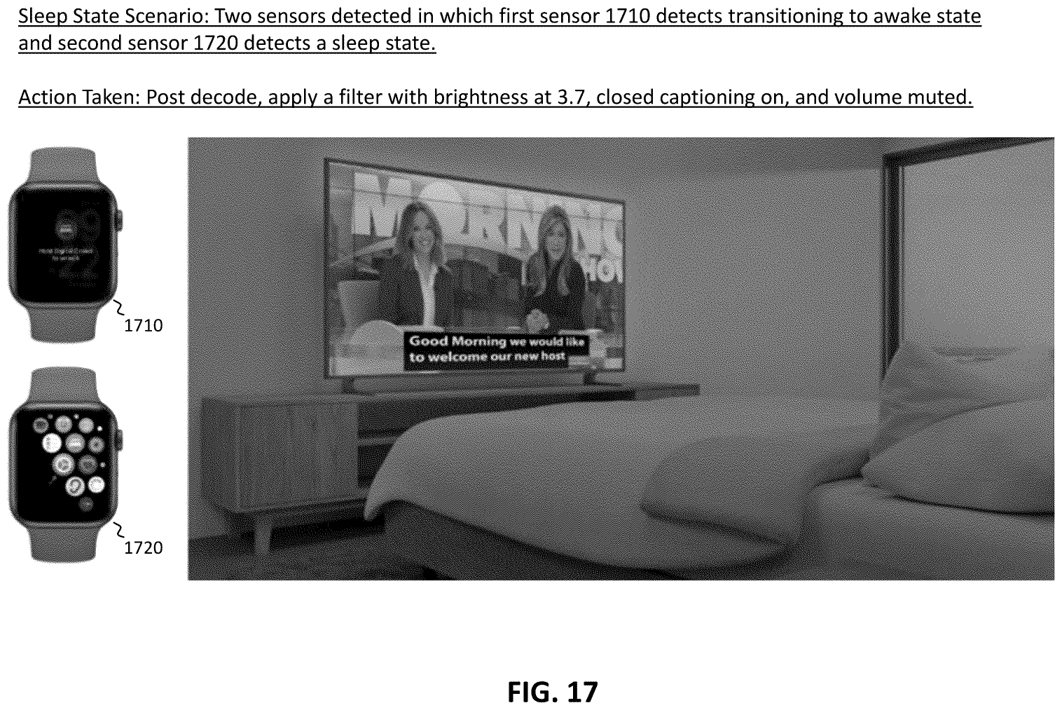

FIG. 17 depicts an example of a scenario in which two sensors are detected and each sensor reports a different sleep state, in accordance with some embodiments of the disclosure. In this embodiment, the control circuitry 220 and/or 228 receives data from multiple sensors, each located within a separate electronic device, such as electronic devices 1710 and 1720.

The data received may be processed by the control circuitry 220 and/or 228 and associated with a sleep state, such as in FIG. 16. As depicted, the sleep state determined based on data from a first sensor associated with device 1710 may indicate that the user associated with the device 1710 is transitioning to an awake state. This may be, for example, a user waking up from long sleep or a nap. Likewise, the sleep state determined based on data from a second sensor associated with device 1720 may indicate that the user associated with the device 1720 is asleep. This may be, for example, the second user associated with device 1720 going from a tired to a sleepy state, or a sleepy state to a deep sleep state.

When the change in sleep state is detected for users associated with devices 1710 and/or 1720, the control circuitry 220 and/or 228, in some embodiments, applies a filter at the processing device. If the processing device does not have the capability, the control circuitry 220 and/or 228 may transmit the sleep state data to a content item server and apply the filter at the content item server.

Since the two sleep states of users associated with devices 1710 and/or 1720 are different, i.e., awake and asleep, the control circuitry 220 and/or 228 detecting a conflict, or opposing sleep states, may select a filter that is a) a midway approach, b) suitable for the first user, c) suitable for the second user, d) a filter that accommodates both the first and the second user, or e) based on priority given between the first and second user. The approaches are further described in the description related to FIG. 13.

Using any of the approaches, the control circuitry 220 and/or 228 may determine that the filter to be applied is to include an increase in brightness at level of 3.7, closed captioning turned on, and volume muted.

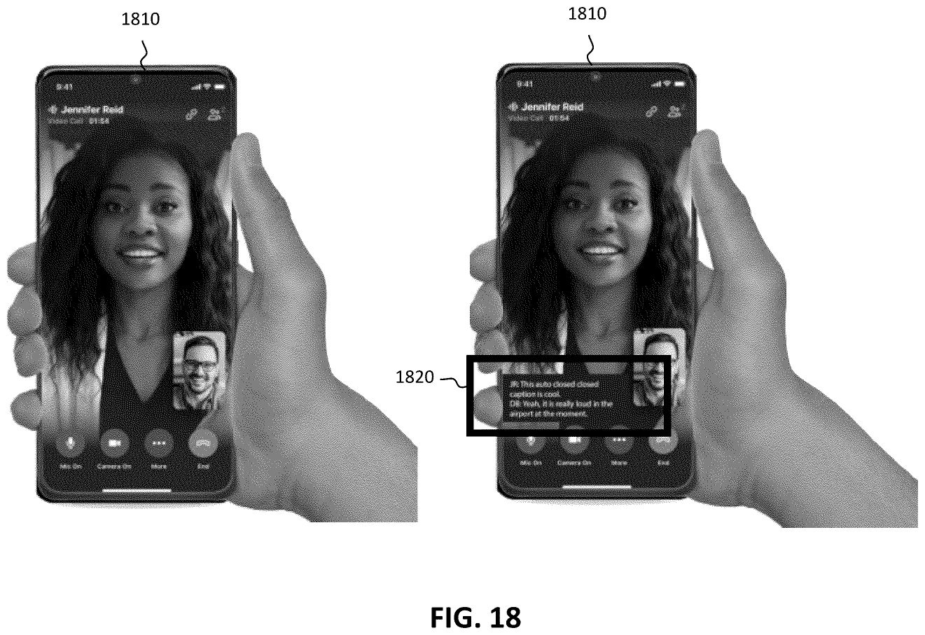

FIG. 18 depicts a conference call in which filters are applied based on a detected sleep state, in accordance with some embodiments of the disclosure. In some embodiments, the process 100 of FIG. 1 may be applied in a conference call setting.

In some embodiments, control circuitry 220 and/or 228 may detect a multiple-sensor environment or multiple devices, each with a set of sensors. In other words, the control circuitry 220 and/or 228 may detect that multiple users are present within a predetermined distance of a display device which is used for a conference call, such as in the same room as the display device.

In this embodiment, the control circuitry 220 and/or 228 receives data from multiple sensors (both devices not shown), each located within a separate electronic device. The data received from the multiple sensors associated with multiple devices may be processed by the control circuitry 220 and/or 228 and associated with a sleep state.

In some embodiments, the control circuitry 220 and/or 228 may determine that the sleep state of a first user associated with device 1810 is different from the sleep state of a second user that is present within a predetermined distance of a display device, such as in the same room as the display device. For example, the second user may be asleep while the first user is awake.

In some embodiments, the first user may receive a conference call while the second user is asleep. So as not to disturb the second user, the control circuitry 220 and/or 228, via a processing device associated with the display device, such as a mobile phone, may apply a filter.

The filter applied, also referred to as conference filter, in some embodiments, may turn off the ringtone for the conference call and switch it to vibrate or only display that a conference call is being requested on the display of the mobile device without any haptic sound.

The filter applied, in some embodiments, may switch to displaying a virtual background or an avatar of the user associated with device 1810 and turn off video such that, respecting the privacy of the surroundings, the room in which the device is located, or the second user, is not shown to other participants of the conference call.

The filter applied, in some embodiments, such as filter 1820, may turn on closed captioning to prevent any sound from the video conference call resulting in disturbing the second user.

The filter applied, in some embodiments, may invoke an artificial intelligence (AI) engine executing an artificial intelligence algorithm. By using the AI algorithm, the control circuitry 220 and/or 228 may automatically respond to any queries directed at the user associated with device 1810 without user input. Alternatively, the control circuitry 220 and/or 228 may automatically present one or more auto responses for the user to select for responding to the query. Such auto-responses, either based on AI, machine learning, or predetermined responses based on the second user's sleep state, allow the first user to communicate to other participants in the conference call without speaking and disturbing the second user.

In another embodiment associated with a conference call, a filter may be applied to block out external noise or ambient noise. In this embodiment, a conference call may be in progress between a first user using the first device 1810 and a plurality of other users (not shown). If the first user's microphone picks up external noise or ambient noise that is near the device 1810, then a filter may be automatically applied to block out external noise or ambient noise such that the other users in the conference are not disturbed by the external noise or ambient noise near the first user. For example, if a dog is barking and that noise is picked up by the first device 1810, then the automatic filter may block that noise other users in the conference are not disturbed by the dog barking noise.

In one embodiment, the filter applied in this scenario may be a mute filter that blocks external noise or ambient noise. In another embodiment, the filter applied in this scenario may be a mute filter and a closed caption filter such that the mute filter blocks external noise or ambient noise and the closed caption filter allows the user associated with device 1810 to read textual translation of speech during the conference call.

Although a few examples of filters for a conference call have been described, the embodiments are not so limited, and additional types of filters may also be applied.

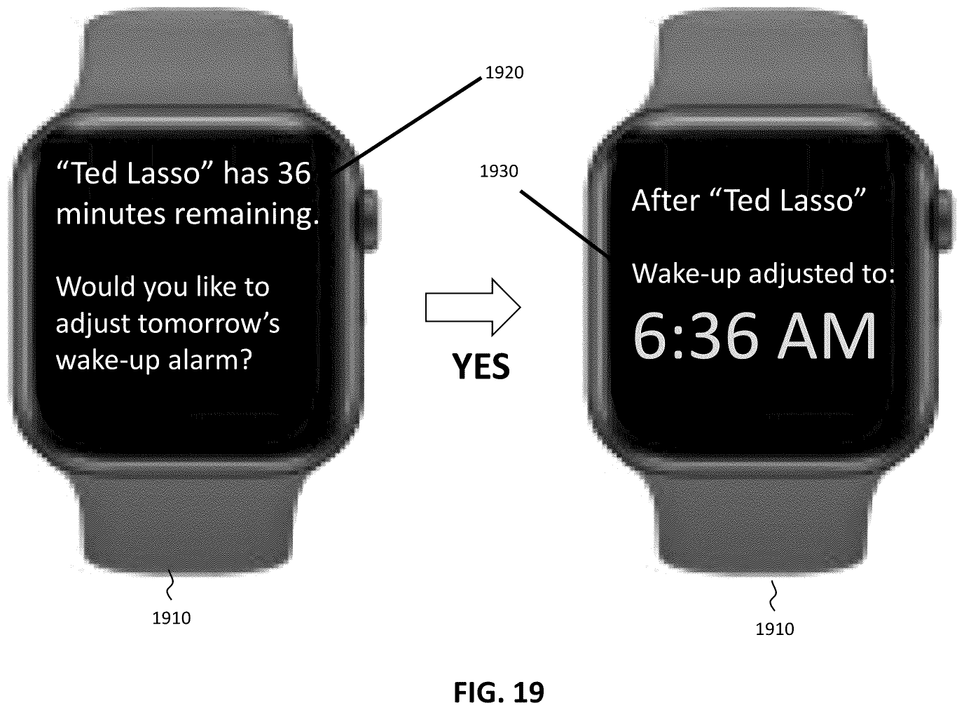

FIG. 19 depicts an adjustment made to a scheduled alarm based on a detected sleep state, in accordance with some embodiments of the disclosure. In some embodiments, the process 100 of FIG. 1 may be applied in a wake-up alarm setting.

In one embodiment, in a single user, single sensor environment, or a set of sensors in a single device environment, the control circuitry 220 and/or 228 may detect a sleep state of a user associated with device 1910. In this embodiment, the control circuitry 220 and/or 228 receives data from a sensor associated with device 1910 and associates the data with a sleep state.

As depicted, the user associated with the device 1910 may be consuming a program, such as an episode of “Ted Lasso,” which has 36 minutes remaining for the program to end, as depicted at 1920. In this embodiment, the user associated with the device 1910 may have had an alarm set up for 6:00 AM for the following morning. Detecting that the user may be staying up late for an additional 36 minutes, due to the user consuming “Ted Lasso,” the control circuitry 220 and/or 228, via the processing device associated with device 1910, may apply a filter that adjusts the wake-up alarm so as to delay it by another 36 minutes, as depicted at 1930. By making the adjustments, the control circuitry 220 and/or 228 may allow the user to sleep an additional 36 minutes to get the proper sleep the user associated with the device 1910 gets on a routine basis. The control circuitry 220 and/or 228 may apply the filter automatically or may do so after the user agrees with the suggestion to delay the wake-up alarm.

In some embodiments, the control circuitry 220 and/or 228 may determine that the show or program consumed by the user is part of a series or is a show that is broadcasted daily at the same time. Determining the user's interest in the program and determining that the program is broadcasted daily, certain days of the week, or at a certain time, the control circuitry 220 and/or 228 may automatically change the user's schedule for all future occurrences when the program is to be displayed. In other words, determining that the user is likely to stay up late to consume the rest of the series because the user has consumed the first episode, or a threshold number of episodes, the control circuitry may adjust the wake-up time for the days when such program is broadcasted or streamed. The control circuitry may apply the filter to change all future alarms on the program days automatically or may do so after the user agrees with the suggestion to delay the wake-up alarm.

In another embodiment, another filter may be applied to the alarm in a multi-user setting. For example, if a determination is made by the control circuitry that a second user who is also present in the room will be asleep at the time of the wake-up alarm, then the control circuitry may switch the alarm from audio to a haptic or vibration mode.

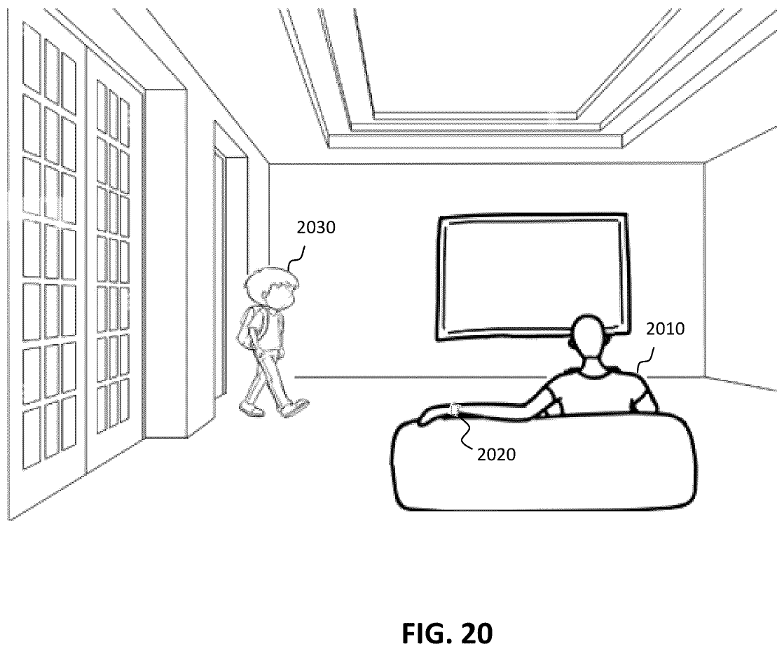

FIG. 20 is an example of applying a filter applied based on parental control settings, in accordance with some embodiments of the disclosure. In some embodiments, the process 100 of FIG. 1 may be applied in a parental control setting.

In this embodiment, a filter is selected based on a sleep state of a user as well as based on any parental control preferences of the user. In one embodiment, the control circuitry 220 and/or 228 may detect a sleep state of a user 2010 associated with device 2020.

In this embodiment, the control circuitry 220 and/or 228 receives data from sensor associated with device 2020 and associates the data with a sleep state. The data received by the control circuitry, in one embodiment, may be associated with an awake state for the user 2010. What is considered an awake state may be defined by the control circuitry 220 and/or 228, such as a user being awake for a predetermined period of time, such as for two hours etc. The data may vary for what is considered as an awake state and be based on the user and a case-by-case basis. For example, a user awake for 30 minutes may be considered to be in an awake state while for another user being awake for one hour would be considered to be in an awake state. The data may vary based on, for example, the user's sleep patterns, their age, demographic, medical condition, or amount of work performed by the user prior to going to sleep.

Once the sleep state is detected, the filter, in some embodiments, for this state of sleep may include using the highest level of brightness and volume and being displayed without closed captioning. The control circuitry may also obtain user preferences from the user's profile in selecting a filter.

While the user 2010 is consuming the media asset with the applied filter, the control circuitry may detect that a child 2030 has walked into the room. The control circuitry may accordingly apply another filter that incorporates a parental setting. The control circuitry may apply the filter based on preferences in the user's profile relating to parental controls for when the user consumes the media asset in the presence of a child. The control circuitry may also apply the filter automatically based on parental controls that may be obtained from crowdsourcing data from other parents. The control circuitry may also look at historical parental controls applied by the user when consuming the media asset in the presence of a child and use the same setting when it detects that a child has walked in while the user was consuming the media asset.

For example, if the content item is deemed inappropriate for a child, the control circuitry may apply a filter that removes sound, fast-forwards through inappropriate content, applies blackout areas for certain content item, or switches to a blank screen. The control circuitry may also mute bad language or display blank spaces or another word for the bad language in a closed caption. The control circuitry may also apply a filter that pauses the media asset until the child leaves the room or until the parent approves its display.

The detection that a child has walked in may be made by the control circuitry 220 and/or 228 based on detecting a device signal, such as a Wi-Fi connection, of a smart watch worn by the child 2030. The detection that a child has walked in the room may also be made by the control circuitry 220 and/or 228 by using a camera associated with the display device on which the media asset is being consumed, such as a smart television set, and applying facial recognition to determine that the person who entered the room is a child.

In addition to the scenario described in FIG. 20, a filter using parental controls may also be applied in other scenarios. For example, in a multiple-sensor scenario where a first sensor is associated with a device worn by a user and a second sensor is associated with a device worn by a child, the control circuitry may apply a parental filter based on the different sleep states of the adult and the child. For example, the child may be given priority when sleeping so that a filter suitable for the child's sleep state may be applied and given priority over the adult's sleep state.

Although a sleep state of a person has been used for determining the filter to be applied, the described embodiments are not so limited, and filters may also be applied based on determined physical and/or behavioral characteristics. For example, such physical and/or behavioral characteristics may include a person is actively engaged and the other is disengaged, bothered, or distracted. In another example, behavioral characteristics may include a pet is acting erratically based on a display or an output and as such a filter is to be applied to calm down the pet in a same room as the display or output device.

In addition to sleep states, other states such as emotional states, conditional states, physical states, may also be used to determine a filter. Such states may be determined based on biometrics obtain for a user. For example, if a sad emotional state is detected, then a filter that hides sad content in a media asset may be applied. Likewise, if a happy emotional state is detected, then a filter that continue to keep the user in the happy emotional state may be applied.

It will be apparent to those of ordinary skill in the art that methods involved in the above-described embodiments may be embodied in a computer program product that includes a computer-usable and/or readable medium. For example, such a computer-usable medium may consist of a read-only memory device, such as a CD-ROM disk or conventional ROM device, or a random-access memory, such as a hard drive device or a computer diskette, having a computer-readable program code stored thereon. It should also be understood that methods, techniques, and processes involved in the present disclosure may be executed using processing circuitry.

The processes discussed above are intended to be illustrative and not limiting. Only the claims that follow are meant to set bounds as to what the present invention includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

1. A method comprising:
detecting a plurality of sensors within a predetermined distance of an output device;
obtaining biometric data from each of the detected plurality of sensors;
determining, based on the obtained biometric data, a sleep state of a first user and a second user associated with the detected plurality of sensors;
selecting a filter based on the determined sleep states; and
applying the selected filter to a content item, prior to the content item being outputted on the output device.

2. The method of claim 1, wherein selecting the filter based on the determined sleep states further comprises:
determining a first sleep state for the first user associated with a first sensor, from the detected plurality of sensors, based on the obtained biometric data for the first sensor;
determining a second sleep state for the second user associated with a second sensor, from the detected plurality of sensors, based on the obtained biometric data for the second sensor;
determining that the first sleep state is different from the second sleep state; and
selecting the filter that accommodates both the first sleep state and the second sleep state.

3. The method of claim 2, wherein the filter that accommodates both the first sleep state and the second sleep state is a filter that a) applies one or more of brightness, volume, closed captioning, or white noise settings to the content item or b) removes ambient noise from the content item.

4. The method of claim 2, further comprising:
determining that the first sleep state is an awake sleep state;
determining that the second sleep state is an asleep sleep state; and
applying a filter that mutes sound of the content item and turns on closed captioning based on the determined first and second sleep states.

5. (canceled)

6. The method of claim 1, wherein the filtering of the content item is performed either by a processing device or a content server associated with the output device, and the filtering is performed without changing the controls of the output device.

7. (canceled)

8. The method of claim 1, further comprising:
determining that a processing device associated with the output device cannot perform the filtering of the content item; and
in response to the determination that the processing devices cannot perform the filtering:
transmitting data associated with the determined sleep state to a content server; and
performing the filtering of the content item at the content server.

9-14. (canceled)

15. The method of claim 1, further comprising:
determining that an alarm device is configured to display the content item, wherein the content item is a wake-up alarm set for a predetermined wake-up time;
determining that consumption of the content item, to which the filter is applied, would delay a sleep routine by a predetermined amount of time; and
in response to the determination that the consumption of the content item, to which the filter is applied, would delay the sleep routine by the predetermined amount of time, automatically modifying the wake-up alarm on the alarm device by delaying the wake-up time by the predetermined amount of time.

16. The method of claim 1, further comprising:
determining that a second sleep state associated with a second sensor, from the plurality of sensors, based on the obtained biometric data for the second sensor, is associated with an asleep state, wherein the second sensor is associated with a second user;
determining that a first user associated with the first sensor is receiving the content item while the second user is asleep, wherein the content item is a video conference call; and
in response to the determination, automatically applying the filter to the conference call; and
displaying the conference call with the applied filter.

17. The method of claim 16, wherein the filter applied to the conference call turns off audio associated with the conference call and turns on closed captioning for speech uttered in the conference call.

18. (canceled)

19. The method of claim 1, further comprising:
determining that the first user is engaged in a conference call via a first electronic device having a microphone;
detecting ambient noise being picked up by the microphone of the first electronic device; and
in response to detecting ambient noise being picked up by the microphone of the first electronic device, applying a filter that mutes the microphone.

20. The method of claim 1, wherein the output device is either a display device or an audio device.

21. (canceled)

22. A system comprising:
communications circuitry configured to access a plurality of sensors; and
control circuitry configured to:
detect the plurality of sensors within a predetermined distance of an output device;
obtain biometric data from each of the detected plurality of sensors;
determine, based on the obtained biometric data, a sleep state of a first user and a second user associated with the detected plurality of sensors;
select a filter based on the determined sleep states; and
apply the selected filter to a content item, prior to the content item being outputted on the output device.

23. The system of claim 22, wherein selecting the filter based on the determined sleep states further comprises, the control circuitry configured to:
determine a first sleep state for the first user associated with a first sensor, from the detected plurality of sensors, based on the obtained biometric data for the first sensor;
determine a second sleep state for the second user associated with a second sensor, from the detected plurality of sensors, based on the obtained biometric data for the second sensor;
determine that the first sleep state is different from the second sleep state; and
select the filter that accommodates both the first sleep state and the second sleep state.

24. The system of claim 23, wherein the filter that accommodates both the first sleep state and the second sleep state is a filter that a) applies one or more of brightness, volume, closed captioning, or white noise settings to the content item or b) removes ambient noise from the content item.

25. The system of claim 23, further comprising, the control circuitry configured to:
determine that the first sleep state is an awake sleep state;
determine that the second sleep state is an asleep sleep state; and
apply a filter that mutes sound of the content item and turns on closed captioning based on the determined first and second sleep states.

26. (canceled)

27. The system of claim 22, wherein the filtering of the content item is performed either by a processing device or a content server associated with the output device, and the filtering is performed without changing the controls of the output device.

28. (canceled)

29. The system of claim 22, further comprising, the control circuitry configured to:
determine that a processing device associated with the output device cannot perform the filtering of the content item; and
in response to the determination that the processing devices cannot perform the filtering:
transmit data associated with the determined sleep state to a content server; and
perform the filtering of the content item at the content server.

30-35. (canceled)

36. The system of claim 22, further comprising, the control circuitry configured to:
determine that an alarm device is configured to display the content item, wherein the content item is a wake-up alarm set for a predetermined wake-up time;
determine that consumption of the content item, to which the filter is applied, would delay a sleep routine by a predetermined amount of time; and
in response to the determination that the consumption of the content item, to which the filter is applied, would delay the sleep routine by the predetermined amount of time, automatically modify the wake-up alarm on the alarm device by delaying the wake-up time by the predetermined amount of time.

37. The system of claim 22, further comprising, the control circuitry configured to:
determine that a second sleep state associated with a second sensor, from the plurality of sensors, based on the obtained biometric data for the second sensor, is associated with an asleep state, wherein the second sensor is associated with a second user;
determine that a first user associated with the first sensor is receiving the content item while the second user is asleep, wherein the content item is a video conference call; and
in response to the determination, automatically apply the filter to the conference call; and
display the conference call with the applied filter.

38. The system of claim 37, wherein the filter applied to the conference call turns off audio associated with the conference call and turns on closed captioning for speech uttered in the conference call.

39. (canceled)

40. The system of claim 22, further comprising, the control circuitry configured to:
determine that the first user is engaged in a conference call via a first electronic device having a microphone;
detect ambient noise being picked up by the microphone of the first electronic device; and
in response to detecting ambient noise being picked up by the microphone of the first electronic device, apply a filter that mutes the microphone.

41. The system of claim 22, wherein the output device is either a display device or an audio device.

42. (canceled)
